(19) United States
(12) Patent Application Publication
(10) Pub. No.: US 2015/0297629 A1
(43) Pub. Date: Oct. 22, 2015

(54) MODULATION OF RENIN-ANGIOTENSIN SYSTEM (RAS) RELATED DISEASES BY ANGIOTENSINOGEN

(71) Applicant: Isis Pharmaceuticals, Inc,, Carlsbad (CA)

(72) Inventors: Adam Mullick, Carlsbad, CA (US); Mark J. Graham, San Clemente, CA (US); Rosanne M. Crooke, Carlsbad, CA (US)

(73) Assignee: Isis Pharmaceuticals, Inc., Carlsbad, CA (US)

(21) Appl. No.: 14/417,762

(22) PCT Filed: Jul. 26, 2013
(86) PCT No.: PCT/US2013/052399

§ 371 Date: Jan. 27, 2015

Related U.S. Application Data
(60) Provisional application No. 61/676,858, filed on Jul. 27, 2012.

(51) Int. Cl. A61K 31/7125; A61K 9/00; A61K 45/06; C12N 15/113

(52) U.S. Cl. CPC A61K 31/7125; C12N 15/113; A61K 9/0019; A61K 45/06; C12N 2310/11; C12N 2310/3525; C12N 2310/341; C12N 2310/315; C12N 2310/3341

(57) ABSTRACT

Disclosed herein are antisense compounds and methods for modulating AGT and modulating a RAS pathway related disease, disorder or condition in an individual in need thereof. RAS related diseases in an individual such as hypertension or organ damage can be treated, ameliorated or prevented with the administration of antisense compounds targeted to AGT.

### SEQUENCE LISTING

The present application is being filed along with a Sequence Listing in electronic format.

The Sequence Listing is provided as a file entitled BIOL0205WOSEQ.txt created Jul. 26, 2013, which is 100 kb in size. The information in the electronic format of the sequence listing is incorporated herein by reference in its entirety.

### FIELD OF THE INVENTION

The present invention provides methods, compounds, and compositions for modulating a RAS pathway related disease by administering an angiotensinogen modulator to an animal. The present invention also provides methods, compounds, and compositions for modulating hypertension and organ damage by administering an angiotensinogen inhibitor to an animal.

### BACKGROUND OF THE INVENTION

Angiotensinogen (AGT), also known as SERPINA8 or ANHU, is a member of the serpin family and is a component of the renin-angiotensin system (RAS) pathway (also known as the renin-angiotensin-aldosterone system (RAAS)). It is primarily produced in the liver and is released into the circulation where renin converts it into angiotensin I. Angiotensin I is subsequently converted into angiotensin II by angiotension converting enzyme (ACE). Angiotensin II is a peptide hormone which causes vasoconstriction which, in turn, can increase blood pressure. Angiotensin II also stimulates secretion of the hormone aldosterone from the adrenal cortex. Aldosterone causes the kidneys to increase reabsorption of sodium and water leading to an increase of the fluid volume in a body which, in turn, can increase blood pressure. Over stimulation or activity of the RAS pathway can lead to high blood pressure. Chronic high blood pressure is known as hypertension. The high blood pressure in a hypertensive subject requires the heart to work harder to circulate blood through the blood vessels.

The World Health Organization (WHO) has identified hypertension as a leading cause of cardiovascular morbidity. Hypertension is a major risk factor for various disease, disorders and conditions such as shortened life expectancy, chronic kidney disease, stroke, myocardial infarction, heart failure, aneurysms of the blood vessels (e.g. aortic aneurysm), peripheral artery disease, heart damage (e.g., heart enlargement or hypertrophy) and other cardiovascular related diseases, disorders and/or conditions.

The prevelance of resistant hypertension (RHTN), hypertension resistant to drug treatment, has steadily increased in number likely due to an ageing population and an ever increasing incidence of obesity. The current projection of approximately 10 million RHTN adults in the United States is expected to continue to rise.

Anti-hypertensive drugs, renal denervation, baroreceptor activation therapy, diet changes and lifestyle changes may reduce hypertension and reduce the diseases, disorders and/or conditions associated with hypertension (Paulis et al., Nat Rev Cardiol, 2012, 9:276-285). However, there are limitations to the therapies currently approved for treating hypertension as a significant subset of all hypertensive patients do not achieve adequate blood pressure control. For example, drugs such as ACE inhibitors and angiotensin receptor blockers (ARBs) that target parts of the RAS pathway are limited in their ability to inhibit the RAS pathway (Nobakht et al., Nat Rev Nephrol, 2011, 7:356-359).

Accordingly, there is a need to find alternative treatments to inhibit the RAS pathway and treat hypertension, especially resistant hypertension. Antisense technology is emerging as an effective means for reducing the expression of certain gene products. Certain early antisense oligonucleotides targeting AGT have provided limited benefit (WO 1997/33623). Compositions provided herein are uniquely useful for therapeutic applications for the modulation of AGT.

### SUMMARY OF THE INVENTION

Provided herein are methods, compounds, and compositions for modulating levels of AGT mRNA and/or protein in an animal. Provided herein are methods, compounds, and compositions for modulating levels of AGT mRNA and/or protein in an animal in order to modulate a RAS pathway related disease, disorder and/or condition in the animal. Also provided herein are methods, compounds, and compositions for identifying an animal having or at risk for a RAS related disease, disorder and/or condition and administering a therapeutically effective amount of a compound targeting AGT to the animal for: ameliorating the RAS related disease, disorder and/or condition in the animal; treating the animal at risk for the RAS related disease, disorder and/or condition; inhibiting AGT expression in the animal suffering from the RAS related disease, disorder and/or condition; reducing the risk of the RAS related disease, disorder and/or condition in the animal; treating RAS dependent organ damage in an animal suffering from a RAS pathway related disease. In certain embodiments, the AGT modulator or compound targeting AGT is an AGT specific inhibitor.

In certain embodiments, AGT specific inhibitors decrease levels of AGT mRNA and/or protein. In certain embodiments, AGT specific inhibitors are nucleic acids, proteins, or small molecules. In certain embodiments, the nucleic acid is an antisense oligonucleotide. In certain embodiments, the antisense oligonucleotide is a modified antisense oligonucleotide.

In certain embodiments, an animal having or at risk for a RAS related disease, disorder and/or condition is treated by selecting the animal having, or at risk for, the RAS related disease, disorder and/or condition and administering to the animal a therapeutically effective amount of a compound comprising a modified oligonucleotide consisting of 12 to 30 linked nucleosides. The modified oligonucleotide can be complementary to an AGT nucleic acid as shown in any of SEQ ID NOs: 1-16.

In certain embodiments, an animal having or at risk for a RAS related disease, disorder and/or condition is treated by selecting the animal having or at risk for the RAS related disease, disorder and/or condition and administering to the animal a therapeutically effective amount of a compound comprising a modified oligonucleotide consisting of 12 to 30 linked nucleosides and having a nucleobase sequence comprising at least 8 contiguous nucleobases complementary to a target segment or target region of SEQ ID NOs: 1-16 as described herein.

In certain embodiments, the AGT modulation occurs in a cell, tissue, organ or organism. In certain embodiments, the cell, tissue or organ is in an animal. In certain embodiments, the animal is a human. In certain embodiments, AGT mRNA levels are reduced. In certain embodiments, AGT protein levels are reduced. Such reduction can occur in a time-dependent manner or in a dose-dependent manner.

Also provided are methods, compounds, and compositions useful for preventing, treating, and ameliorating RAS related diseases, disorders, and conditions.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“About” means within ±10% of a value. For example, if it is stated “the compounds inhibited AGT by at least about 70%”, it is implied that the AGT levels are inhibited within a range of 63% to 77%.

“ACE escape”, also known as angiotensin II reactivation, refers to the inability of currently available ACE inhibitor treatment to reliably suppress plasma angiotensin II levels. The increase in plasma angiotensin II levels during ACE inhibition occurs via other enzymes converting angiotensin I to angiotensin. This incomplete blockage of angiotensin II levels prevents the ACE inhibitors from effectively treating some hypertensive subjects. Angiotensin Receptor Blockers (ARBs) may also be susceptible to ACE escape as other receptors besides the AT1 receptor engage angiotensin metabolites.

“Active pharmaceutical agent” or “pharmaceutical agent” means the substance or substances in a pharmaceutical composition that provide a therapeutic benefit when administered to an individual. For example, in certain embodiments an antisense oligonucleotide targeted to AGT is an active pharmaceutical agent.

“Active target region” or “target region” means a region to which one or more active antisense compounds is targeted.

“Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be co-extensive.

“Administering” means providing a pharmaceutical agent to an individual, and includes, but is not limited to administering by a medical professional and self-administering.

“Aldosterone escape” or “aldosterone breakthrough” refers to the inability of currently available ACE inhibitor Angiotensin Receptor Blocker (ARB) and/or Direct Renin Inhibitor (DRI) treatment to reliably suppress aldosterone release in some treated subjects. This incomplete blockage of aldosterone prevents the ACE inhibitors, DRIs and ARBs from effectively treating some hypertensive subjects.

“Amelioration” refers to a lessening of at least one indicator, sign, or symptom of an associated disease, disorder, or condition. In certain embodiments, amelioration includes a delay or slowing in the progression of one or more indicators of a condition or disease. The severity of indicators may be determined by subjective or objective measures, which are known to those skilled in the art. For example, amelioration of hypertension can be assessed by measuring systolic and diastolic blood pressure.

“Angiotensinogen” and “AGT” is used interchangeably herein. Angiotensinogen is also known as SERPINA8 and ANHU.

“Angiotensinogen nucleic acid” or “AGT nucleic acid” means any nucleic acid encoding AGT. For example, in certain embodiments, an AGT nucleic acid includes a DNA sequence encoding AGT, an RNA sequence transcribed from DNA encoding AGT (including genomic DNA comprising introns and exons), and an mRNA sequence encoding AGT. “AGT mRNA” means an mRNA encoding an AGT protein.

“AGT specific inhibitor” refers to any agent capable of specifically inhibiting the expression of AGT mRNA and/or AGT protein at the molecular level. For example, AGT specific inhibitors include nucleic acids (including antisense compounds such as RNasH, siRNA and blockmer antisense compounds), peptides, antibodies, small molecules, and other agents capable of specifically inhibiting the expression of AGT mRNA and/or AGT protein. In certain embodiments, by specifically modulating AGT mRNA level and/or AGT protein expression, AGT specific inhibitors can affect components of the renin-angiotensin system (RAS) pathway. In certain embodiments, by specifically modulating AGT mRNA level and/or AGT protein expression, AGT specific inhibitors can affect RAS pathway related diseases, disorders and/or conditions such as blood pressure. Similarly, in certain embodiments, AGT specific inhibitors can affect other molecular processes in an animal.

“Animal” refers to a human or non-human animal, including, but not limited to, mice, rats, rabbits, dogs, cats, pigs, and non-human primates, including, but not limited to, monkeys and chimpanzees.

“Anti-hypertensive drug” refers to a drug capable of lowering blood pressure. Examples of such drugs include, but are not limited to, RAS inhibitors, diuretics, calcium channel blockers, adrenergic receptor antagonists, adrenergic agonists and vasodilators. In one example, the anti-hypertensive drug captopril can be used in combination with the AGT compound described herein to treat an animal having or at risk of having a RAS pathway related disease, disorder and/or condition.

“Anti-hypertensive procedure” refers to a medical procedure performed on a subject to reduce hypertension. Examples of such procedures include renal denervation and baroreceptor activation therapy,

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Blood pressure” refers to the pressure of the blood in the circulatory system against the walls of the blood vessel. The blood pressure is due mainly to the beating of the heart in an animal. During each heartbeat, the blood pressure varies between a maximum (systolic) blood pressure (SBP) and minimum (diastolic) blood pressure (DBP). The mean arterial pressure (MAP) is the average arterial pressure during a heartbeat cycle. Blood pressure can be measure by a blood pressure meter (i.e., a sphygromanometer). Normal blood pressure at rest is within the range of 100-140 mmHg systolic and 60-90 mmHg diastolic and is commonly expressed as the systolic pressure (top reading)/diastolic pressure (bottom reading) mmHg.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses concomitant, parallel or sequential administration.

“Complementarity” means the capacity for pairing between nucleobases of a first nucleic acid and a second nucleic acid.

“Constrained ethyl” or “cEt” refers to a bicyclic nucleoside having a furanosyl sugar that comprises a methyl(methyleneoxy) (4′-CH(CH₃)—O-2′) bridge between the 4′ and the 2′ carbon atoms.

“Contiguous nucleobases” means nucleobases immediately adjacent to each other.

“Diluent” means an ingredient in a composition that lacks pharmacological activity, but is pharmaceutically necessary or desirable. For example, the diluent in an injected composition may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors. In an example, an effective amount of an AGT antisense oligonucleotide decreases blood pressure and/or ameliorates organ damage due to hypertension.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap segment” and the external regions may be referred to as “wing segments.”

“Hybridization” means the annealing of complementary nucleic acid molecules. In certain embodiments, complementary nucleic acid molecules include an antisense compound and a target nucleic acid.

“Hypertension” or “HTN” refers to a chronic medical condition where the blood pressure in an animal is elevated. The elevated blood pressure requires the heart to work harder to circulate blood through the blood vessels. High blood pressure is said to be present if it is persistently at or above 140/90 mmHg. Hypertension is classified as primary (essential) or secondary. Primary hypertension has no clear cause and is thought to be linked to genetics, diet, lack of exercise and obesity. Secondary hypertension is caused by another medical condition. Hypertension is a major risk factor for shortened life expectancy, chronic kidney disease, stroke, myocardial infarction, heart failure, aneurysms of the blood vessels (e.g. aortic aneurysm), peripheral artery disease, organ damage (e.g., heart enlargement or hypertrophy) and other cardiovascular diseases, disorders and/or conditions or symptoms thereof. Anti-hypertensive drugs, diet changes and lifestyle changes may reduce hypertension and reduce the diseases, disorders and/or conditions associated with hypertension. Hypertension can be nonresistant to drug intervention (i.e., controllable by commercially available drug therapies) or resistant to drug intervention.

“Identifying an animal having, or at risk for, a RAS related disease, disorder and/or condition” means identifying an animal having been diagnosed with a RAS related disease, disorder and/or condition or identifying an animal predisposed to develop a RAS related disease, disorder and/or condition. Individuals predisposed to develop a RAS related disease, disorder and/or condition include, for example, individuals with a familial history a RAS related disease such as hypertension. Such identification may be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Internucleoside linkage” refers to the chemical bond between nucleosides.

“Linked nucleosides” means adjacent nucleosides which are bonded together.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage and/or modified nucleobase. A “modified nucleoside” means a nucleoside having a modified sugar moiety and/or modified nucleobase.

“Modified oligonucleotide” means an oligonucleotide comprising a modified internucleoside linkage, a modified sugar and/or a modified nucleobase.

“Modified sugar” refers to a substitution or change from a natural sugar.

“Modulating” refers to changing or adjusting a feature in a cell, tissue, organ or organism. For example, modulating AGT mRNA can mean to increase or decrease the level of AGT mRNA and/or AGT protein in a cell, tissue, organ or organism. Modulating AGT mRNA and/or protein can lead to an increase or decrease in a RAS related disease, disorder and/or condition in a cell, tissue, organ or organism. A “modulator” effects the change in the cell, tissue, organ or organism. For example, an AGT antisense compound can be a modulator that increases or decreases the amount of AGT mRNA and/or AGT protein in a cell, tissue, organ or organism.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nonresistant hypertension”, “nonrefractory hypertension” or “controlled hypertension” is defined as hypertension that responds to treatment resulting in, for example, blood pressure <140 mmHg SBP or <90 mmHg DBP with concurrent use of up to 3 anti-hypertensive agents.

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleotide” means a nucleoside having a phosphate group covalently linked to the sugar portion of the nucleoside.

“Organ damage” or “end organ damage” refers to damage occurring in major organs fed by the circulatory system such as the heart (e.g., heart muscle hypertrophy, reduced heart function and/or heart failure), kidney (e.g., albuminurea, proteinurea, reduced renal function and/or renal failure), eyes (e.g., hypertensive retinopathy), brain (e.g., stroke) and the like. The organs can be damaged by hypertension in an animal. In certain embodiments, the heart damage is fibrosis, heart cell and/or muscle hypertrophy leading to heart enlargement.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutical composition” means a mixture of substances suitable for administering to an individual. For example, a pharmaceutical composition may comprise one or more active pharmaceutical agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset, development or progression of a disease, disorder, or condition for a period of time from minutes to indefinitely. Prevent also means reducing the risk of developing a disease, disorder, or condition.

“Renin-angiotensin system”, “Renin-angiotensin system pathway”, “RAS pathway” or “RAS” refer to a multi-component enzymatic pathway where a precursor component (angiotensinogen) is converted by various enzymes such as renin and enzyme angiotensin-converting-enzyme (ACE) into downstream components such as angiotensin I and angiotensin II. Angiotensin I stimulates secretion of the steroid aldosterone in the pathway. Various components of this pathway have been targeted by agonists or antagonists to block the production of the components. For example renin inhibitors, ACE inhibitors, angiotensin-receptor blockers (ARBs) and the like have been developed to inhibit or block the RAS pathway. However, commercially available therapies targeting various RAS pathway components have been ineffective in completely inhibiting or blocking the RAS pathway due to various mechanisms (Nobakht et al., Nat Rev Nephrol, 2011, 7:356-359).

“RAS related disease, disorder and/or condition” or “RAS pathway related disease, disorder and/or condition” refers to any disease, disorder or condition related to RAS or RAAS (Renin-Angiotensin-Aldosterone System) in an animal. Examples of RAS related diseases, disorders and/or conditions include shortened life expectancy, hypertension (e.g. nonresistant hypertension, resistant hypertension), kidney disease (e.g., chronic kidney disease, polycystic kidney disease), stroke, heart disease (e.g., myocardial infarction, heart failure, valvular heart disease), aneurysms of the blood vessels (e.g. aortic aneurysm), peripheral artery disease, organ damage (e.g., heart damage or hypertrophy), tissue fibrosis and other cardiovascular diseases, disorders and/or conditions or symptoms thereof. In certain embodiments, RAS related disease, disorder and/or condition does not include hypertension.

“Resistant hypertension” or “RHTN” is defined as (1) blood pressure ≧140 mmHg SBP or ≧90 mmHg DBP despite concurrent use of 3 anti-hypertensive agents from different drug classes or (2) use of ≧4 anti-hypertensive drugs regardless of blood pressure.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments. In an example, an antisense compound is specifically hybridizable to a target when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” refers to administering a pharmaceutical composition to an animal in order to effect an alteration or improvement of a disease, disorder, or condition in the animal. In certain embodiments, one or more pharmaceutical compositions can be administered to the animal.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleotide) or a DNA nucleotide (i.e. 3-D-deoxyribonucleotide).

### Certain Embodiments

In certain embodiments, provided are compounds specifically modulating AGT. In certain embodiments, the AGT specific modulators are AGT specific inhibitors, for use in treating, preventing, or ameliorating a RAS related disease, disorder and/or condition. In certain embodiments, AGT specific inhibitors are nucleic acids (including antisense compounds), peptides, antibodies, small molecules, and other agents capable of inhibiting the expression of AGT mRNA and/or AGT protein. In certain embodiments, the AGT specific inhibitors are antisense oligonucleotides. In certain embodiments, the antisense oligonucleotides are modified antisense oligonucleotides.

In certain embodiments, the compounds target an AGT nucleic acid. In certain embodiments, the AGT nucleic acid is any of the human sequences set forth in GENBANK Accession No. NM_{—}000029.3 (incorporated herein as SEQ ID NO: 1), GENBANK Accession No. CR606672.1 (incorporated herein as SEQ ID NO: 2), GENBANK Accession No. AK307978.1 (incorporated herein as SEQ ID NO: 3), GENBANK Accession No. AK303755.1 (incorporated herein as SEQ ID NO: 4), GENBANK Accession No. AK293507.1 (incorporated herein as SEQ ID NO: 5), and the complement of the nucleotides 24354000 to 24370100 of GENBANK Accession No. NT_{—}167186.1 (incorporated herein as SEQ ID NO: 6). In certain embodiments, the AGT nucleic acid is any of the mouse sequences set forth in GENBANK Accession NM_{—}007428.3 (incorporated herein as SEQ ID NO: 7), GENBANK Accession W13136.1 (incorporated herein as SEQ ID NO: 8), GENBANK Accession AI785758.1 (incorporated herein as SEQ ID NO: 9), GENBANK Accession BG863202.1 (incorporated herein as SEQ ID NO: 10), the complement of GENBANK Accession BF020372.1 (incorporated herein as SEQ ID NO: 11), and the complement of the nucleotides 51911800 to 51928000 of GENBANK Accession No. NT_{—}078575.6 (incorporated herein as SEQ ID NO: 12). In certain embodiments, the AGT nucleic acid is any of the rat sequences set forth in GENBANK Accession NM_{—}134432.2 (incorporated herein as SEQ ID NO: 13), GENBANK Accession CV104009.1 (incorporated herein as SEQ ID NO: 14), GENBANK Accession BF549490.1 (incorporated herein as SEQ ID NO: 15), and the complement of the nucleotides 17172500 to Ser. No. 17/188,250 of GENBANK Accession NW_{—}047536.2 (incorporated herein as SEQ ID NO: 16).

In certain embodiments, the compounds targeting AGT described herein are for use in modulating AGT expression. In certain embodiments, the AGT modulators are AGT specific inhibitors. In certain embodiments, the AGT specific inhibitors decrease AGT expression and/or the RAS pathway. In certain embodiments, the compounds targeting AGT inhibit the RAS pathway by at least 70%, 75%, 80%, 85%, 90%, 95% or 100%.

In certain embodiments, the compounds described herein are for use in inhibiting AGT expression in an animal having, or at risk of having, a RAS pathway related disease, disorder and/or condition comprising selecting the animal suffering from the RAS pathway related disease, disorder or condition and, administering a compound targeting AGT to the animal, wherein the compound administered to the animal inhibits AGT expression in the animal having or at risk for having the RAS pathway related disease, disorder and/or condition.

In certain embodiments, the compounds described herein are for use in treating an animal having, or at risk of having, a RAS pathway related disease, disorder and/or condition comprising selecting the animal having, or at risk of having, the RAS pathway related disease, disorder or condition, and administering a therapeutically effective amount of a compound targeting AGT to the animal, wherein the compound administered to the animal treats the animal having, or at risk of having, the RAS pathway related disease, disorder and/or condition, in the animal.

In certain embodiments, the compounds described herein are for use in treating an animal having, or at risk of having, RAS dependent organ damage comprising selecting an animal suffering from a RAS pathway related disease, disorder and/or condition, and administering a therapeutically effective amount of a compound targeting AGT to the animal, wherein the compound administered to the animal treats and/or reverses the RAS dependent end organ damage in the animal with the RAS pathway related disease, disorder or condition. In certain embodiments the RAS pathway related disease, disorder or condition is hypertension. In certain embodiments the end organ damage is heart muscle hypertrophy.

In certain embodiments, the compounds described herein are for use in treating an animal having or at risk for a RAS pathway related disease comprising selecting the animal having or at risk for a RAS pathway related disease, and administering to the animal a therapeutically effective amount of a compound comprising a modified oligonucleotide consisting of 12 to 30 linked nucleosides, wherein the modified antisense oligonucleotide is complementary to an AGT nucleic acid as shown in any of SEQ ID NOs: 1-16, and wherein the compound administered to the animal treats the animal having or at risk for having the RAS pathway related disease.

In certain embodiments, the RAS pathway related disease, disorder or condition is shortened life expectancy, hypertension, kidney disease (e.g., chronic kidney disease), stroke, cardiac disease (e.g., myocardial infarction, heart failure, valvular heart disease), aneurysms of the blood vessels, peripheral artery disease, organ damage and other cardiovascular diseases, disorders and/or conditions or symptoms thereof. In certain embodiments, the hypertension is nonresistant hypertension or resistant hypertension. In certain embodiments, the aneurysm of the blood vessels is aortic aneurysm. In certain embodiments, the organ damage is heart muscle hypertrophy or fibrosis in an organ or tissue. In certain embodiments, the organ is heart, liver or kidney and the tissue is derived from the heart, liver or kidney. In certain embodiments, the RAS pathway related disease, disorder or condition is not hypertension.

In certain embodiments, the compounds described herein are for use in treating nonresistant hypertension comprising selecting the animal having or at risk for nonresistant hypertension, and administering to the animal a therapeutically effective amount of a compound comprising a modified oligonucleotide consisting of 12 to 30 linked nucleosides, wherein the modified antisense oligonucleotide is complementary to an AGT nucleic acid as shown in any of SEQ ID NOs: 1-16, and wherein the compound administered to the animal treats the animal having or at risk of having nonresistant hypertension. In certain embodiments the modified oligonucleotide is administered in combination with one or more anti-hypertensive drugs.

In certain embodiments, the compounds described herein are for use in treating resistant hypertension comprising selecting the animal having or at risk for resistant hypertension, and administering to the animal a therapeutically effective amount of a compound comprising a modified oligonucleotide consisting of 12 to 30 linked nucleosides, wherein the modified antisense oligonucleotide is complementary to an AGT nucleic acid as shown in any of SEQ ID NOs: 1-16, and wherein the compound administered to the animal treats the animal having or at risk of having resistant hypertension. In certain embodiments the modified oligonucleotide is administered in combination with one or more anti-hypertensive drugs.

In certain embodiments, provided are methods, compounds, and compositions for modulating a symptom or marker of a RAS pathway related disease, disorder and/or condition. In certain embodiments, the marker can be selected from one or more of shortened life expectancy, hypertension, chronic kidney disease, stroke, myocardial infarction, heart failure, valvular heart disease, aneurysms of the blood vessels, peripheral artery disease, organ damage and other cardiovascular diseases, disorders and/or conditions or symptoms thereof.

In certain embodiments, the compounds for use in the methods comprise an antisense oligonucleotide comprising a nucleobase sequence at least 80%, at least 85%, at least 90%, at least 95%, at least 96%, at least 97%, at least 98%, or at least 99% complementary to an equal length portion of SEQ ID NOs: 1-16. In certain embodiments, the compound may comprise a modified oligonucleotide comprising a nucleobase sequence 100% complementary to an equal length portion of SEQ ID NOs: 1-16.

In certain embodiments, the compounds for use in the methods comprise an antisense oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19 or at least 20 contiguous nucleobases of a nucleobase sequence complementary to any of the sequences recited in SEQ ID NOs: 1-16.

In certain embodiments, the compounds for use in the methods comprise an antisense oligonucleotide consisting of 12 to 30 linked nucleosides. In certain embodiments, the modified oligonucleotide consists of 12, 13, 14, 15, 16, 17, 18, 19, 20, 21, 22, 23, 24, 25, 26, 27, 28, 29 or 30 linked nucleosides.

In certain embodiments, the compounds for use in the methods consist of a single-stranded modified oligonucleotide.

In certain embodiments, the compounds for use in the methods comprise at least one modified internucleoside linkage. In certain embodiments, the modified internucleoside linkage is a phosphorothioate internucleoside linkage. In certain embodiments, each modified internucleoside linkage is a phosphorothioate internucleoside linkage.

In certain embodiments, the compounds for use in the methods comprise at least one nucleoside comprising a modified sugar. In certain embodiments, the modified sugar is a bicyclic sugar. In certain embodiments, the modified sugar comprises a 2′-O-methoxyethyl (2′MOE).

In certain embodiments, the compounds for use in the methods comprise at least one nucleoside comprising a modified nucleobase. In certain embodiments, the modified nucleobase is a 5-methylcytosine.

In certain embodiments, the compounds for use in the methods comprise a modified antisense oligonucleotide comprising: (i) a gap segment consisting of linked deoxynucleosides; (ii) a 5′ wing segment consisting of linked nucleosides; (iii) a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In certain embodiments, the compounds for use in the methods comprise a modified antisense oligonucleotide comprising: (i) a gap segment consisting of eight to sixteen linked deoxynucleosides; (ii) a 5′ wing segment consisting of two to six linked nucleosides; (iii) a 3′ wing segment consisting of two to six linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the compounds for use in the methods comprise a modified antisense oligonucleotide comprising: (i) a gap segment consisting often linked deoxynucleosides; (ii) a 5′ wing segment consisting of five linked nucleosides; (iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the animal is a human.

In certain embodiments, the compounds described herein are administered to an animal to treat, prevent or ameliorate a RAS pathway related disease, disorder or condition. In certain embodiments, administration to an animal is by a parenteral route. In certain embodiments, the parenteral administration is any of subcutaneous or intravenous administration. In certain embodiments, the compound is administered to the animal at most once a day, at most once a week, at most once every two weeks, at most once every month, at most once a quarter, at most once every half year, at most once every year, at most once every five years or at most once every ten years.

In certain embodiments, the compound is co-administered with one or more second agent(s). The compound of the invention and one or more second agent can be administered concomitantly or sequentially.

In certain embodiments the second agent is an anti-hypertensive drug, a procedure to decrease hypertension, a diet change and/or a lifestyle change.

Examples of the anti-hypertensive drug include, but are not limited to, any of a RAS inhibitor, diuretic, calcium channel blocker, adrenergic receptor antagonist, adrenergic agonist and vasodilator.

In certain embodiments, the compound or oligonucleotide is in salt form.

In certain embodiments, the compounds or compositions are formulated with a pharmaceutically acceptable carrier or diluent.

In certain embodiments, provided is the use of a compound targeting AGT as described herein in the manufacture of a medicament. In certain embodiments, provided is the use of a compound targeting AGT as described herein for treating, preventing, or ameliorating a RAS pathway related disease, disorder and/or condition as described herein. In certain embodiments, the compound is an AGT specific inhibitor, for use in treating, preventing, or ameliorating a RAS related disease, disorder and/or condition. In certain embodiments, the AGT specific inhibitor is a nucleic acid (including antisense compound), peptide, antibody, small molecule, or other agent capable of inhibiting the expression of AGT mRNA and/or AGT protein. In certain embodiments, the AGT specific inhibitor is an antisense oligonucleotide. In certain embodiments, the antisense oligonucleotide is a modified antisense oligonucleotide. In certain embodiments, the AGT has a sequence as shown in any of SEQ ID NOs: 1-16.

In certain embodiments, the AGT specific inhibitor is used to reduce AGT expression. The AGT compound can be used in combination therapy with one or more additional agent or therapy as described herein. Agents or therapies can be administered concomitantly or sequentially to an animal.

In certain embodiments, provided is a kit for treating, preventing, or ameliorating a RAS pathway related disease and/or condition, disease, disorder or condition, wherein the kit comprises: (i) an AGT specific inhibitor as described herein; and optionally (ii) an additional agent or therapy as described herein.

A kit of the present invention may further include instructions for using the kit to treat, prevent, or ameliorate a RAS pathway related disease, disorder or condition as described herein.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound can be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to AGT nucleic acid is 10 to 30 nucleotides in length. In other words, antisense compounds are from 10 to 30 linked nucleobases. In other embodiments, the antisense compound comprises a modified oligonucleotide consisting of 8 to 80, 10 to 80, 12 to 50, 15 to 30, 18 to 24, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values. In some embodiments, the antisense compound is an antisense oligonucleotide.

In certain embodiments, the antisense compound comprises a shortened or truncated modified oligonucleotide. The shortened or truncated modified oligonucleotide can have a single nucleoside deleted from the 5′ end (5′ truncation), the central portion or alternatively from the 3′ end (3′ truncation). A shortened or truncated oligonucleotide can have two or more nucleosides deleted from the 5′ end, two or more nucleosides deleted from the central portion or alternatively can have two or more nucleosides deleted from the 3′ end. Alternatively, the deleted nucleosides can be dispersed throughout the modified oligonucleotide, for example, in an antisense compound having one or more nucleoside deleted from the 5′ end, one or more nucleoside deleted from the central portion and/or one or more nucleoside deleted from the 3′ end.

In certain embodiments, the antisense compound comprises a lengthened or long modified oligonucleotide. When a single additional nucleoside is present in a lengthened oligonucleotide, the additional nucleoside can be located at the 5′ end, 3′ end or central portion of the oligonucleotide. When two or more additional nucleosides are present, the added nucleosides can be adjacent to each other, for example, in an oligonucleotide having two nucleosides added to the 5′ end (5′ addition), to the 3′ end (3′ addition) or the central portion, of the oligonucleotide. Alternatively, the added nucleoside can be dispersed throughout the antisense compound, for example, in an oligonucleotide having one or more nucleoside added to the 5′ end, one or more nucleoside added to the 3′ end, and/or one or more nucleoside added to the central portion.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to an AGT nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound can optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2′-modified nucleosides (such 2′-modified nucleosides can include 2′-MOE, and 2′-O—CH₃, among others), and bicyclic sugar modified nucleosides (such bicyclic sugar modified nucleosides can include those having a 4′-(CH2)n-O-2′ bridge, where n=1 or n=2). Each distinct region comprises uniform sugar moieties or comprises different types of sugar moieties. The wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In some embodiments, the sugar moieties in the X wing segment are the same. In some embodiments, the types of sugar moieties in the X wing segment are the different. In some embodiments, the sugar moieties in the Z wing segment are the same. In some embodiments, the types of sugar moieties in the Z wing segment are the different.

In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6, 5-8-5, 1-8-1, 2-6-2, 6-8-6, 5-8-5, 1-8-1, 2-6-2, 2-13-2, 1-8-2, 2-8-3, 3-10-2, 1-18-2, or 2-18-2.

In certain embodiments, the antisense compound as a “wingmer” motif, having a wing-gap or gap-wing configuration, i.e. an X—Y or Y—Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to an AGT nucleic acid possess a 5-10-5 gapmer motif. In certain embodiments, antisense compounds targeted to an AGT nucleic acid possess a 3-14-3 gapmer motif. In certain embodiments, antisense compounds targeted to an AGT nucleic acid possess a 2-13-5 gapmer motif.

In certain embodiments, an antisense compound targeted to an AGT nucleic acid has a gap-widened motif. In certain embodiments, a gap-widened antisense oligonucleotide targeted to an AGT nucleic acid has a gap segment of fourteen 2′-deoxyribonucleosides positioned immediately adjacent to and between wing segments of three chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to an AGT nucleic acid has a gap segment of thirteen 2′-deoxyribonucleosides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

Nucleotide sequences that encode human AGT include, without limitation, the following: GENBANK Accession No. NM_{—}000029.3 (incorporated herein as SEQ ID NO: 1), GENBANK Accession No. CR606672.1 (incorporated herein as SEQ ID NO: 2), GENBANK Accession No. AK307978.1 (incorporated herein as SEQ ID NO: 3), GENBANK Accession No. AK303755.1 (incorporated herein as SEQ ID NO: 4), GENBANK Accession No. AK293507.1 (incorporated herein as SEQ ID NO: 5), and the complement of the nucleotides 24354000 to 24370100 of GENBANK Accession No. NT_{—}167186.1 (incorporated herein as SEQ ID NO: 6).

Nucleotide sequences that encode mouse AGT include, without limitation, the following: GENBANK Accession NM_{—}007428.3 (incorporated herein as SEQ ID NO: 7), GENBANK Accession W13136.1 (incorporated herein as SEQ ID NO: 8), GENBANK Accession AI785758.1 (incorporated herein as SEQ ID NO: 9), GENBANK Accession BG863202.1 (incorporated herein as SEQ ID NO: 10), the complement of GENBANK Accession BF020372.1 (incorporated herein as SEQ ID NO: 11), and the complement of the nucleotides 51911800 to 51928000 of GENBANK Accession No. NT_{—}078575.6 (incorporated herein as SEQ ID NO: 12). In certain embodiments, the AGT nucleic acid is any of the rat sequences set forth in GENBANK Accession NM_{—}134432.2 (incorporated herein as SEQ ID NO: 13), GENBANK Accession CV104009.1 (incorporated herein as SEQ ID NO: 14), GENBANK Accession BF549490.1 (incorporated herein as SEQ ID NO: 15), and the complement of the nucleotides 17172500 to Ser. No. 17/188,250 of GENBANK Accession NW_{—}047536.2 (incorporated herein as SEQ ID NO: 16).

It is understood that the sequence set forth in each SEQ ID NO in the examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for AGT can be obtained by accession number from sequence databases such as NCBI and such information is incorporated herein by reference. In certain embodiments, a target region may encompass the sequence from a 5′ target site of one target segment within the target region to a 3′ target site of another target segment within the target region.

In certain embodiments, a “target segment” is a smaller, sub-portion of a target region within a nucleic acid. For example, a target segment can be the sequence of nucleotides of a target nucleic acid to which one or more antisense compound is targeted. “5′ target site” refers to the 5′-most nucleotide of a target segment. “3′ target site” refers to the 3′-most nucleotide of a target segment.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in AGT mRNA levels are indicative of inhibition of AGT expression. Reductions in levels of an AGT protein are also indicative of inhibition of target mRNA levels. Further, phenotypic changes are indicative of inhibition of AGT expression. For example, a decrease in hypertension can be indicative of inhibition of AGT expression. In another example, a decrease in heart size can be indicative of inhibition of AGT expression. In another example, a decrease in angiotensin level can be indicative of inhibition of AGT expression.

### Hybridization

In some embodiments, hybridization occurs between an antisense compound disclosed herein and an AGT nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrook and Russell, Molecular Cloning: A Laboratory Manual, 3^{rd} Ed., 2001). In certain embodiments, the antisense compounds provided herein are specifically hybridizable with an AGT nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as an AGT nucleic acid).

Non-complementary nucleobases between an antisense compound and an AGT nucleic acid may be tolerated provided that the antisense compound remains able to specifically hybridize to a target nucleic acid. Moreover, an antisense compound may hybridize over one or more segments of an AGT nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to an AGT nucleic acid, a target region, target segment, or specified portion thereof. Percent complementarity of an antisense compound with a target nucleic acid can be determined using routine methods. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound may be fully complementary to an AGT nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as an AGT nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as an AGT nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to an AGT nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ (also referred to as constrained ethyl or cEt) and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U. S. A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Serial Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA and (K) vinyl BNA as depicted below.

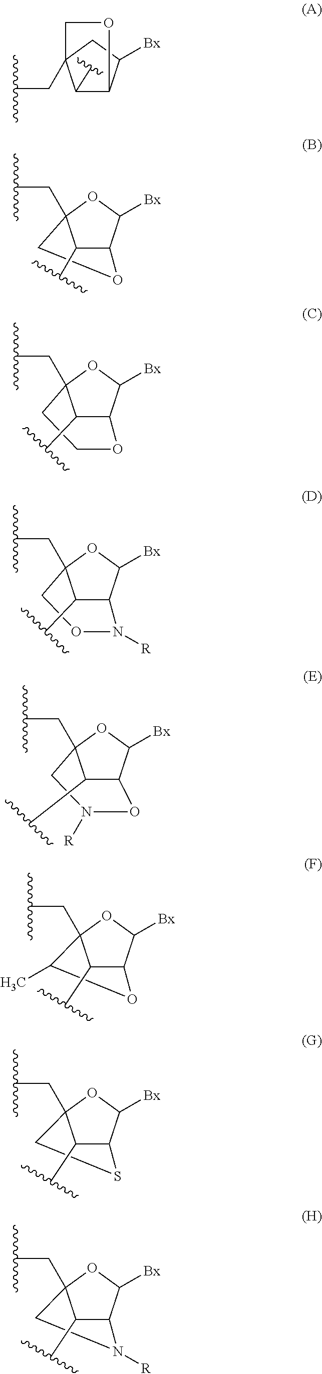

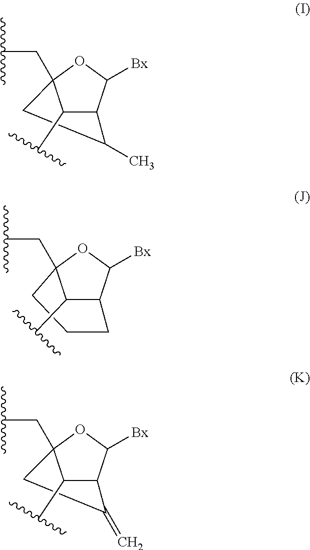

wherein Bx is the base moiety and R is independently H, a protecting group, C₁-C₁₂ alkyl or C₁-C₁₂ alkoxy.

In certain embodiments, bicyclic nucleosides are provided having Formula I:

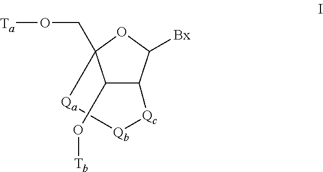

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

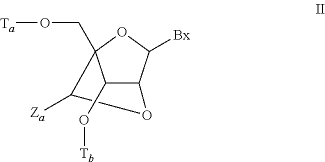

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each Jₑ, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

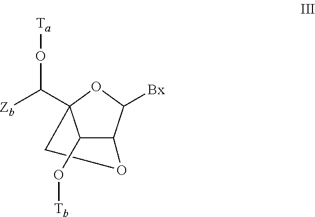

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

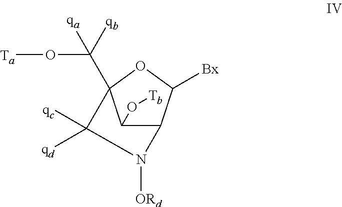

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

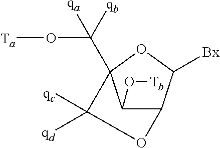

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, q_{c} and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

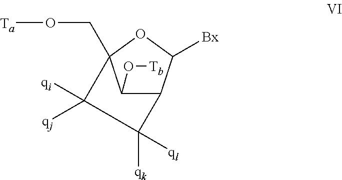

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position.

In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH_{3]2}, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854) or fluoro HNA (F-HNA) having a tetrahydropyran ring system as illustrated below:

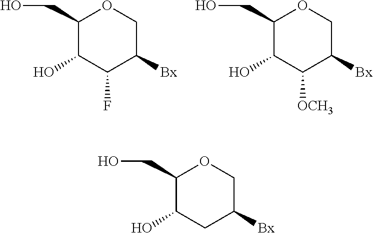

In certain embodiments, sugar surrogates are selected having Formula VII:

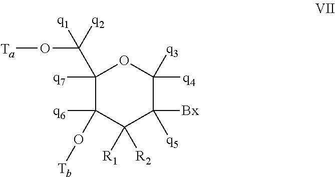

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is methoxyethoxy and R₂ is H.

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following formula:

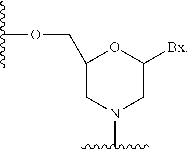

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 published on Aug. 21, 2008 for other disclosed 5′,2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, antisense compounds comprise one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc., 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCT application, WO 06/047842; and Published PCT Application WO 01/049687; the text of each is incorporated by reference herein, in their entirety). Certain modified cyclohexenyl nucleosides have Formula X.

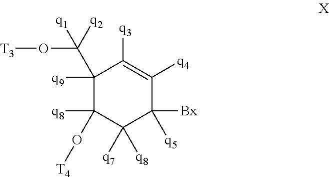

wherein independently for each of said at least one cyclohexenyl nucleoside analog of Formula X:

Bx is a heterocyclic base moiety;

T₃ and T₄ are each, independently, an internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and

q₁, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position of the sugar ring.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854). Such ring systems can undergo various additional substitutions to enhance activity.

Methods for the preparations of modified sugars are well known to those skilled in the art. Some representative U.S. patents that teach the preparation of such modified sugars include without limitation, U.S.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to an AGT nucleic acid comprise one or more modified nucleobases. In certain embodiments, gap-widened antisense oligonucleotides targeted to an AGT nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides can be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to an AGT nucleic acid can be utilized in pharmaceutical compositions by combining the antisense compound with a suitable pharmaceutically acceptable diluent or carrier.

In certain embodiments, the “pharmaceutical carrier” or “excipient” is a pharmaceutically acceptable solvent, suspending agent or any other pharmacologically inert vehicle for delivering one or more nucleic acids to an animal. The excipient can be liquid or solid and can be selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients, which do not deleteriously react with nucleic acids, suitable for parenteral or non-parenteral administration can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to an AGT nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer (e.g., PBS). In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers.

### Dosing

In certain embodiments, pharmaceutical compositions are administered according to a dosing regimen (e.g., dose, dose frequency, and duration) wherein the dosing regimen can be selected to achieve a desired effect. The desired effect can be, for example, reduction of AGT or the prevention, reduction, amelioration or slowing the progression of a disease, disorder or condition associated with AGT or the RAS pathway.

In certain embodiments, the variables of the dosing regimen are adjusted to result in a desired concentration of pharmaceutical composition in a subject. “Concentration of pharmaceutical composition” as used with regard to dose regimen can refer to the compound, oligonucleotide, or active ingredient of the pharmaceutical composition. For example, in certain embodiments, dose and dose frequency are adjusted to provide a tissue concentration or plasma concentration of a pharmaceutical composition at an amount sufficient to achieve a desired effect.

Dosing is dependent on severity and responsiveness of the disease state to be treated, with the course of treatment lasting from several days to several months, or until a cure is effected or a diminution of the disease state is achieved. Dosing is also dependent on drug potency and metabolism. In certain embodiments, dosage is from 0.01 μg to 100 mg per kg of body weight, or within a range of 0.001 mg to 1000 mg dosing, and may be given once or more daily, weekly, monthly, quarterly or yearly, or even once every 2 to 20 years. Following successful treatment, it may be desirable to have the patient undergo maintenance therapy to prevent the recurrence of the disease state, wherein the oligonucleotide is administered in maintenance doses, ranging from 0.01 μg to 100 mg per kg of body weight, once or more daily, once or more weekly, once or more monthly, once or more quarterly, once or more yearly, to once every 20 years or ranging from 0.001 mg to 1000 mg dosing. In certain embodiments, it may be desirable to administer the oligonucleotide from at most once daily, once weekly, once monthly, once quarterly, once yearly, once every two years, once every three years, once every four years, once every five years, once every ten year, to once every 20 years.

### Administration

The compounds or pharmaceutical compositions of the present invention can be administered in a number of ways depending upon whether local or systemic treatment is desired and upon the area to be treated. Administration can be oral, inhaled or parenteral.

In certain embodiments, the compounds and compositions as described herein are administered parenterally. Parenteral administration includes intravenous, intra-arterial, subcutaneous, intraperitoneal or intramuscular injection or infusion; or intracranial, e.g., intrathecal or intraventricular, administration.

In certain embodiments, parenteral administration is by infusion. Infusion can be chronic or continuous or short or intermittent. In certain embodiments, infused pharmaceutical agents are delivered with a pump.

In certain embodiments, parenteral administration is by injection. The injection can be delivered with a syringe or a pump. In certain embodiments, the injection is a bolus injection. In certain embodiments, the injection is administered directly to a tissue or organ.

In certain embodiments, formulations for parenteral, intrathecal or intraventricular administration can include sterile aqueous solutions which can also contain buffers, diluents and other suitable additives such as, but not limited to, penetration enhancers, carrier compounds and other pharmaceutically acceptable carriers or excipients.

In certain embodiments, formulations for oral administration of the compounds or compositions can include, but is not limited to, pharmaceutical carriers, excipients, powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In certain embodiments, oral formulations are those in which compounds provided herein are administered in conjunction with one or more penetration enhancers, surfactants and chelators.

### Conjugated Antisense Compounds

In certain embodiments, the compounds of the invention can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

In certain embodiments, antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of AGT nucleic acids can be tested in vitro in a variety of cell types. Cell types used for such analyses are available from commercial vendors (e.g. American Type Culture Collection, Manassas, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE® (Invitrogen, Carlsbad, Calif.). Antisense oligonucleotide is mixed with LIPOFECTAMINE® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Another reagent used to introduce antisense oligonucleotides into cultured cells includes Cytofectin® (Invitrogen, Carlsbad, Calif.). Antisense oligonucleotide is mixed with Cytofectin® in OPTI-MEM® 1 reduced serum medium (Invitrogen, Carlsbad, Calif.) to achieve the desired concentration of antisense oligonucleotide and a Cytofectin® concentration that typically ranges 2 to 12 ug/mL per 100 nM antisense oligonucleotide.

Another reagent used to introduce antisense oligonucleotides into cultured cells includes Oligofectamine™ (Invitrogen Life Technologies, Carlsbad, Calif.). Antisense oligonucleotide is mixed with Oligofectamine™ in Opti-MEM™-1 reduced serum medium (Invitrogen Life Technologies, Carlsbad, Calif.) to achieve the desired concentration of oligonucleotide with an Oligofectamine™ to oligonucleotide ratio of approximately 0.2 to 0.8 μL per 100 nM.

Another reagent used to introduce antisense oligonucleotides into cultured cells includes FuGENE 6 (Roche Diagnostics Corp., Indianapolis, Ind.). Antisense oligomeric compound was mixed with FuGENE 6 in 1 mL of serum-free RPMI to achieve the desired concentration of oligonucleotide with a FuGENE 6 to oligomeric compound ratio of 1 to 4 μL of FuGENE 6 per 100 nM.

Another technique used to introduce antisense oligonucleotides into cultured cells includes electroporation (Sambrook and Russell in Molecular Cloning. A Laboratory Manual. Third Edition. Cold Spring Harbor laboratory Press, Cold Spring Harbor, N.Y. 2001).

Cells are treated with antisense oligonucleotides by routine methods. Cells are typically harvested 16-24 hours after antisense oligonucleotide treatment, at which time RNA or protein levels of target nucleic acids are measured by methods known in the art and described herein (Sambrook and Russell in Molecular Cloning. A Laboratory Manual. Third Edition. Cold Spring Harbor laboratory Press, Cold Spring Harbor, N.Y. 2001). In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art (Sambrook and Russell in Molecular Cloning. A Laboratory Manual. Third Edition. Cold Spring Harbor laboratory Press, Cold Spring Harbor, N.Y. 2001). Antisense oligonucleotides are typically used at concentrations ranging from 1 nM to 300 nM when transfected with LIPOFECTAMINE2000®, Lipofectin or Cytofectin. Antisense oligonucleotides are used at higher concentrations ranging from 625 to 20,000 nM when transfected using electroporation.

### RNA Isolation

RNA analysis can be performed on total cellular RNA or poly(A)+mRNA. Methods of RNA isolation are well known in the art (Sambrook and Russell, Molecular Cloning: A Laboratory Manual, 3^{rd} Ed., 2001). RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of an AGT nucleic acid can be assayed in a variety of ways known in the art (Sambrook and Russell, Molecular Cloning: A Laboratory Manual, 3^{rd} Ed., 2001). For example, target nucleic acid levels can be quantitated by, e.g., Northern blot analysis, competitive polymerase chain reaction (PCR), or quantitative real-time PCR. RNA analysis can be performed on total cellular RNA or poly(A)+mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM® 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT, real-time-PCR reactions are carried out by methods well known to those skilled in the art.

Gene (or RNA) target quantities obtained by real time PCR are normalized using either the expression level of a gene whose expression is constant, such as cyclophilin A or GAPDH, or by quantifying total RNA using RIBOGREEN® (Invitrogen, Inc. Carlsbad, Calif.). Cyclophilin A or GAPDH expression is quantified by real time PCR, by being run simultaneously with the target, multiplexing, or separately. Total RNA is quantified using RIBOGREEN® RNA quantification reagent (Invitrogen, Carlsbad, Calif.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to an AGT nucleic acid. Methods for designing real-time PCR probes and primers are well known in the art, and may include the use of software such as PRIMER EXPRESS® Software (Applied Biosystems, Foster City, Calif.).

The PCR probes can have JOE or FAM covalently linked to the 5′ end and TAMRA or MGB covalently linked to the 3′ end, where JOE or FAM is the fluorescent reporter dye and TAMRA or MGB is the quencher dye. In some cell types, primers and probe designed to a sequence from a different species are used to measure expression. For example, a human GAPDH primer and probe set can be used to measure GAPDH expression in monkey-derived cells and cell lines.

Gene target quantities obtained by RT, real-time PCR can be normalized using either the expression level of GAPDH, a gene whose expression is constant, or by quantifying total RNA using RiboGreen™ (Molecular Probes, Inc. Eugene, Oreg.). GAPDH expression can be quantified by RT, real-time PCR, by being run simultaneously with the target, multiplexing, or separately. Total RNA can be quantified using RiboGreen™ RNA quantification reagent (Molecular Probes, Inc. Eugene, Oreg.).

### Analysis of Protein Levels

Antisense inhibition of AGT nucleic acids can be assessed by measuring AGT protein levels. Protein levels of AGT can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS) (Sambrook and Russell, Molecular Cloning: A Laboratory Manual, 3^{rd} Ed., 2001). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of human and rat AGT are commercially available.

### In Vivo Testing of Antisense Compounds

Antisense compounds, for example, antisense oligonucleotides, are tested in animals to assess their ability to inhibit expression of AGT and/or the RAS pathway and produce phenotypic changes such as a decrease in one or more RAS pathway related diseases. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency depends upon factors such as route of administration and animal body weight. In one embodiment, following a period of treatment with antisense oligonucleotides, RNA is isolated from liver tissue and changes in AGT nucleic acid expression are measured. Changes in AGT protein levels can also be measured. Changes in AGT expression can also be measured by determining the level of inhibition of the RAS pathway. RAS pathway related diseases, disorders and/or conditions may be used as markers for determining the level of AGT inhibition.

### Certain Indications

In certain embodiments, the invention provides methods of treating an individual comprising administering one or more pharmaceutical compositions of the present invention. In certain embodiments, the individual has, or is at risk for, a RAS pathway related disease, disorder or condition. In certain embodiments the invention provides methods for prophylactically reducing AGT expression in an individual. Certain embodiments include treating an individual in need thereof by administering to an individual a therapeutically effective amount of an antisense compound targeted to an AGT nucleic acid.

In certain embodiments, administration of a therapeutically effective amount of an antisense compound targeted to an AGT nucleic acid is accompanied by monitoring of AGT levels in the serum or tissue of an individual, to determine an individual's response to administration of the antisense compound. An individual's response to administration of the antisense compound is used by a physician to determine the amount and duration of therapeutic intervention.

In certain embodiments, administration of an antisense compound targeted to an AGT nucleic acid results in reduction of AGT expression by at least 15, 20, 25, 30, 35, 40, 45, 50, 55, 60, 65, 70, 75, 80, 85, 90, 95, 99% or 100% or a range defined by any two of these values. In certain embodiments, administration of an antisense compound targeted to an AGT nucleic acid results in inhibition of the RAS pathway by at least 15, 20, 25, 30, 35, 40, 45, 50, 55, 60, 65, 70, 75, 80, 85, 90, 95, 99% or 100% or a range defined by any two of these values. In certain embodiments, administration of an antisense compound targeted to an AGT nucleic acid results in a change the RAS pathway related disease, disorder, condition, symptom or marker (e.g., hypertension or organ damage). In certain embodiments, administration of an AGT antisense compound increases or decreases the RAS related disease, disorder, condition, symptom or marker by at least 15, 20, 25, 30, 35, 40, 45, 50, 55, 60, 65, 70, 75, 80, 85, 90, 95, 99% or 100% or a range defined by any two of these values.

In certain embodiments, pharmaceutical compositions comprising an antisense compound targeted to AGT are used for the preparation of a medicament for treating a patient suffering or susceptible to a RAS related disease, disorder or condition.

In certain embodiments, the methods described herein include administering a compound comprising a modified oligonucleotide having an 8, 9, 10, 11, 12, 13, 14, 15, 16, 17, 18, 19 or 20 contiguous nucleobase portion complementary to AGT.

### Certain Combination Therapies

In certain embodiments, a first agent comprising an antisense compound provided herein is co-administered with one or more secondary agents. In certain embodiments, the antisense compound is an antisense oligonucleotide. In certain embodiments, the antisense oligonucleotide is a modified oligonucleotide.

In certain embodiments, such second agents are designed to treat the same RAS pathway related disease, disorder or condition as the first agent described herein. In certain embodiments, such second agents are designed to treat a different disease, disorder, or condition as the first agent described herein. In certain embodiments, such second agents are designed to treat an undesired side effect of one or more pharmaceutical compositions as described herein. In certain embodiments, such first agents are designed to treat an undesired side effect of a second agent. In certain embodiments, second agents are co-administered with the first agent to treat an undesired effect of the first agent. In certain embodiments, second agents are co-administered with the first agent to produce a combinational or additive effect. In certain embodiments, second agents are co-administered with the first agent to produce a synergistic effect.

In certain embodiments, the co-administration of the first and second agents permits use of lower dosages than would be required to achieve a therapeutic or prophylactic effect if the agents were administered as independent therapy. In certain embodiments the dose of a co-administered second agent is the same as the dose that would be administered if the second agent was administered alone. In certain embodiments the dose of a co-administered second agent is greater than the dose that would be administered if the second agent was administered alone.

In certain embodiments, a first agent and one or more second agents are administered at the same time. In certain embodiments, the first agent and one or more second agents are administered at different times. In certain embodiments, the first agent and one or more second agents are prepared together in a single pharmaceutical formulation. In certain embodiments, the first agent and one or more second agents are prepared separately.

In certain embodiments, second agents include, but are not limited to, certain procedures to reduce hypertension, diet changes, lifestyle changes, anti-fibrotic drugs and anti-hypertensive drugs such as RAS inhibitors, diuretics, calcium channel blockers, adrenergic receptor antagonists, adrenergic agonists and vasodilators.

Examples of procedures that can reduce hypertension include, but are not limited to, renal denervation and baroreceptor activation therapy.

Examples of RAS inhibitors include, but are not limited to ACE inhibitors (e.g., captopril, enalapril, fosinopril, lisinopril, perindopril, quinapril, ramipril, trandolapril and benazepril), angiotensin II receptor antagonists (e.g., candesartan, eprosartan, irbesartan, losartan, olmesartan, telmisartan and valsartan), renin inhibitors (e.g., aliskiren), aldosterone receptor antagonists (e.g., eplerenone and spironolactone).

Examples of diuretics include loop diuretics (e.g., bumetanide, ethacrynic acid, furosemide, torsemide), thiazide diuretics (e.g., epitizide, hydrochlorothiazide, chlorothiazide and bendroflumethiazide), thiazide-like diuretics (e.g., indapamide, chlorthalidone and metolazone) and potassium-sparing diuretics (e.g., amiloride, triamterene and spironolactone).

Examples of calcium channel blockers include dihydropyridines (e.g., amlodipine, felodipine, isradipine, lercanidipine, nicardipine, nifedipine, nimodipine and nitrendipine) and non-dihydropyridines (e.g., diltiazem and verapamil).

Examples of adrenergic receptor antagonists include Beta blockers (e.g., atenolol, metoprolol, nadolol, oxprenolol, pindolol, propranolol and timolol), Alpha blockers (e.g., doxazosin, phentolamine, indoramin, phenoxybenzamine, prazosin, terazosin and tolazoline) and mixed Alpha+Beta blockers (e.g., bucindolol, carvedilol and labetalol).

Examples of vasodilators include sodium nitroprusside and hydralazine and its derivatives.

Examples of adrenergic agonists include alpha-2 agonists (e.g., clonidine, guanabenz, methyldopa and moxonidine).

Additional examples of anti-hypertensive drugs include guanethidine, reserpine and the like.

The second agents can be used in combination with the therapeutic compounds described herein to decrease a RAS pathway related disease, disorder and/or condition such as hypertension, organ damage and the like.

### ADVANTAGES OF THE INVENTION

Provided herein are methods and compositions for the modulation of AGT that can treat, prevent and/or ameliorate a RAS pathway related disease, disorder and/or condition such as hypertension or organ damage. In a particular embodiment, provided are AGT antisense oligonucleotides (antisense oligonucleotides targeting a nucleic acid encoding AGT protein) to treat, prevent and/or ameliorate a RAS pathway related disease, disorder and/or condition or symptoms thereof as described herein.

Currently, commercially available therapies targeting various RAS pathway components have been ineffective in completely inhibiting or blocking the RAS pathway. The mechanism(s) for this ineffective inhibition has not been fully elucidated, but may occur by ACE escape and/or aldosterone escape pathways. The antisense oligonucleotide targeting AGT described herein has been shown to completely block the RAS pathway. Accordingly, AGT inhibition is not susceptible to the ACE escape or aldosterone escape mechanisms confounding the commercially approved therapies for hypertension. Therefore, an AGT antisense oligonucleotide may provide superior therapeutic efficacy over the current commercial therapeutics used to treat, prevent and/or ameliorate hypertension in a subject, especially those subject suffering from resistant hypertension. An extended duration of action and more complete inhibition of tissue RAS activity also contribute to the superiority of an AGT antisense oligonucleotide relative to current commercial therapeutics. Also, the AGT antisense oligonucleotide can be used in combination with commercially available anti-hypertensive medications (e.g., ACE inhibitors and/or ARBs) to provide an additive therapeutic effect.

Additionally, the antisense oligonucleotide targeting AGT has been shown to localize to and decrease AGT in the liver, heart and kidney. An ability to decrease AGT in liver tissue indicates that the oligonucleotide can target and inhibit the main source of AGT production. An ability to decrease AGT in cardiac tissue indicates that the oligonucleotide can be useful in treating cardiac diseases such as myocardial infarction, heart failure, valvular heart disease, heart tissue hypertrophy and the like. An ability to decrease AGT in kidney tissue indicates that the oligonucleotide can be useful in treating kidney related diseases such as hypertension, chronic kidney disease and the like. Reducing AGT, and therefore all angiotensin fragments, in extrahepatic tissues is predicted to be the most efficacious means of limiting pathogenic tissue RAS activity.

Another advantage of the invention is that the antisense oligonucleotide targeting AGT has been shown to reverse cardiac hypertrophy within two weeks of antisense oligonucleotide administration. Hence, an AGT antisense oligonucleotide may be able to reverse organ damage and/or fibrosis caused by hypertension in a subject.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### In Vivo Antisense Inhibition of Murine Angiotensinogen

Sprague-Dawley rats are a multipurpose model used for safety and efficacy evaluations. The effect of antisense inhibition of angiotensinogen with ISIS 552668 was studied in this model.

ISIS 552668 (CACTGATTTTTGCCCAGGAT; SEQ ID NO: 17), which was one of the antisense oligonucleotides tested in the assay, was designed as a 5-10-5 MOE gapmer, and is 20 nucleosides in length, wherein the central gap segment is comprised of ten 2′-deoxynucleosides and is flanked on both sides (in the 5′ and 3′ directions) by wings comprising 5 nucleosides each. Each nucleoside in the 5′ wing segment and each nucleoside in the 3′ wing segment has a 2′-MOE modification. The internucleoside linkages throughout the gapmer are phosphorothioate (P═S) linkages. All cytosine residues throughout the gapmer are 5-methylcytosines. ISIS 552668 is targeted to nucleobases 1679 to 1698 of rat angiotensinogen (GENBANK Accession No. NM_{—}134432.2, incorporated herein as SEQ ID NO: 13).

### Treatment

Groups of Sprague-Dawley rats were injected with 10 mg/kg/week, 20 mg/kg/week, 40 mg/kg/week, or 80 mg/kg/week of ISIS 552668 administered for 4 weeks. A control group of rats was injected with phosphate buffered saline (PBS) administered weekly for 4 weeks. The rats were sacrificed at the end of the study at week 4. Whole liver and kidney were harvested for RNA analysis and plasma was collected for protein analysis.

### Angiotensinogen RNA Analysis

RNA was extracted from liver tissue for real-time PCR analysis of angiotensinogen, using primer probe set RTS3550 (forward sequence AGCACGACTTCCTGACTTGGA, designated herein as SEQ ID NO: 18; reverse sequence TTGTAGGATCCCCGAATTTCC, designated herein as SEQ ID NO: 19; probe sequence AACCCGCCTCCTCGGGCCAT, designated herein as SEQ ID NO: 20). The mRNA levels were normalized using the housekeeping gene, GAPDH. As shown in Table 1, the antisense oligonucleotides achieved significant reduction of angiotensinogen over the PBS control. Results are presented as percent inhibition of angiotensinogen, relative to control. The ED₅₀ for liver and kidney was calculated to be 17.0 and 9.5 respectively.

TABLE 1
Percent inhibition of angiotensinogen mRNA in Sprague-Dawley rats

| Dose | | |
|---|---|---|
| (mg/kg/wk) | Liver | Kidney |
| 10 | 19 | 41 |
| 20 | 59 | 68 |
| 40 | 86 | 75 |
| 80 | 96 | 77 |

### Protein Analysis

Angiotensinogen plasma levels were measured by ELISA (IBL International, Toronto, Canada). As shown in Table 2, antisense inhibition of angiotensinogen by ISIS 552668 resulted in a significant dose-dependent reduction of angiotensinogen protein. Results are presented as percent inhibition of angiotensinogen, relative to PBS control. The ED₅₀ for plasma protein reductions was 23.6 mg/kg.

TABLE 2
Percent inhibition of angiotensinogen protein in Sprague-Dawley rats

| Dose | % |
|---|---|
| (mg/kg/wk) | inhibition |
| 10 | 25 |
| 20 | 38 |
| 40 | 83 |
| 80 | 93 |

### Effect on Blood Pressure

Systolic blood pressure (SBP), mean arterial pressure (MAP), and diastolic blood pressure (DBP) in the rats was measured by the tail-cuff volume-pressure method (Kent Scientific, Torrington, Conn.). The results are presented in Table 3 and indicate that antisense inhibition of angiotensinogen resulted in dose-dependent reductions in all three parameters in the rats

TABLE 3
Blood pressure parameters (mm Hg) in Sprague-Dawley rats
after 4 wks of treatments

| Dose | | | |
|---|---|---|---|
| (mg/kg/wk) | SBP | MAP | DBP |
| PBS | 149 | 125 | 113 |
| 10 | 138 | 117 | 107 |
| 40 | 118 | 92 | 79 |
| 80 | 109 | 82 | 68 |

### Example 2

### In Vivo Antisense Inhibition of Angiotensinogen in Female SHR Rats

Spontaneously hypertensive (SHR) rats are a model used for genetic hypertension and hypertensive drug research (Okamoto, A. K. and Aoki K. Jpn. Circ. J. 1963. 27: 282-293). The effect of antisense inhibition of angiotensinogen with ISIS 552668 was studied in this model.

### Study 1

Groups of female SHR rats were injected with 50 mg/kg/week, 75 mg/kg/week, or 100 mg/kg/week of ISIS 552668 administered for 2 weeks. A control group of female SHR rats was injected with phosphate buffered saline (PBS) administered weekly for 2 weeks. Another control group constituted a group of wild-type rats injected with phosphate buffered saline (PBS) administered weekly for 2 weeks.

### Effect on Blood Pressure

Systolic blood pressure (SBP), mean arterial pressure (MAP), and diastolic blood pressure (DBP) in the rats was measured by the tail-cuff volume-pressure method (Kent Scientific, Torrington, Conn.). The results are presented in Table 4. The results indicate that antisense inhibition of angiotensinogen resulted in dose-dependent reductions in all three parameters in the rats compared to the control group.

TABLE 4
Blood pressure parameters in 2 weeks (mm Hg)

| | Dose of ASO | | | |
|---|---|---|---|---|
| Group | (mg/kg/wk) | SBP | MAP | DBP |
| Wild-type | — | 136 | 100 | 83 |
| control | | | | |
| SHR control | — | 179 | 146 | 131 |
| SHR treated with | 50 | 144 | 114 | 100 |
| ISIS 552668 | 75 | 140 | 111 | 98 |
| | 100 | 122 | 95 | 81 |

### Study 2

Groups of female SHR rats were injected with 50 mg/kg/week, 75 mg/kg/week, or 100 mg/kg/week of ISIS 552668 administered for 2 weeks. A control group of female SHR rats was injected with phosphate buffered saline (PBS) administered weekly for 2 weeks. Another control group constituted a group of wild-type rats injected with phosphate buffered saline (PBS) administered weekly for 2 weeks. After 2 weeks, the rats were treated with Captopril at 50 mg/kg/day and blood pressure was measured after 4 days of this treatment.

### Effect on Blood Pressure

Systolic blood pressure (SBP), mean arterial pressure (MAP), and diastolic blood pressure (DBP) in the rats was measured by the tail-cuff volume-pressure method (Kent Scientific, Torrington, Conn.). The results are presented in Table 5.

TABLE 5
Blood pressure parameters (mm Hg)

| | | Dose of | | | |
|---|---|---|---|---|---|
| | | ASO | | | |
| Group | Captopril | (mg/kg/wk) | SBP | MAP | DBP |
| Wild-type | No | — | 131 | 96 | 79 |
| control | | | | | |
| SHR control | Yes | — | 179 | 146 | 131 |
| SHR rats | Yes | — | 145 | 113 | 97 |
| | Yes | 50 | 127 | 97 | 82 |
| | Yes | 75 | 113 | 87 | 74 |
| | Yes | 100 | 120 | 94 | 81 |

The results indicate that antisense inhibition of angiotensinogen generally resulted in dose-dependent reductions in all three parameters in the rats. The results also indicate that treatment of the rats with ISIS 552668 at doses of 50 mg/kg/week and 75 mg/kg/week before Captopril treatment further decreased blood pressure parameters compared to that with Captopril treatment alone. Hence, additive blood pressure lowering was achieved with the combination of angiotensinogen antisense oligonucleotide and Captopril treatment, suggesting that improvements in efficacy are possible when antisense oligonucleotides are added to existing RAS inhibitor therapeutics. Such improvement in treatments could be desirable in resistant hypertensive subjects or subjects not achieving their blood pressure goal with existing RAS inhibitors and/or patients that experience ACE escape and/or aldosterone breakthrough.

Additionally, Captopril treatment did not result in further blood pressure reductions in rats receiving 100 mg/kg/week of ISIS 552668, demonstrating that maximal inhibition of RAS signaling was already achieved by treatment with ISIS 552668 in this group. The data indicates that complete inhibition of RAS-dependent blood pressure control could be achieved within 2 weeks of ASO treatment.

### Effect on Heart Size

The size of the heart was measured immediately after harvest. The results from each group are presented in Table 6, expressed as a percent of body weight. The results indicate that treatment with ISIS 552668 after 3 weeks reversed the increase in heart size of the SHR rats to that observed in the wild-type controls.

TABLE 6
Heart size (% body weight)

| | | Dose of | |
|---|---|---|---|
| | | ASO | Heart size |
| Group | Captopril | (mg/kg/wk) | (%) |
| Wild-type | No | — | 44 |
| control | | | |
| SHR rats | Yes | — | 47 |
| | Yes | 50 | 46 |
| | Yes | 75 | 45 |
| | Yes | 100 | 42 |

### Study 3

A group of SHR was injected with 100 mg/kg/week of ISIS 552668 administered for 3 weeks. A control group of SHR was injected with phosphate buffered saline (PBS) administered weekly for 3 weeks. Another control group of SHR was injected with 100 mg/kg/week of a control oligonucleotide (a nucleotide not targeted to angiotensinogen) administered for 3 weeks. Another control group of SHR was treated with Captopril at 150 mg/kg/day administered for 3 weeks.

### Effect on Blood Pressure

Mean arterial pressure (MAP) in the rats was measured by the tail-cuff volume-pressure method (Kent Scientific, Torrington, Conn.). The results are presented in Table 7 and indicate that treatment with ISIS 552668 and Captopril reduced MAP levels to comparable levels.

TABLE 7
MAP (mm Hg)

| Group | Week 1 | Week 2 | Week 3 |
|---|---|---|---|
| Control oligonucleotide | 136 | 135 | 146 |
| PBS control | 150 | 136 | 150 |
| Captopril control | 107 | 109 | 112 |
| ISIS 552668 | 117 | 104 | 109 |

RNA Analysis RNA was extracted from liver tissue for real-time PCR analysis of angiotensinogen, using primer probe set RTS3550. The mRNA levels were normalized using the housekeeping gene, GAPDH. As shown in Table 8, ISIS 552668 achieved significant reduction of angiotensinogen over the PBS control. Results are presented as percent inhibition of angiotensinogen, relative to control.

TABLE 8
Percent inhibition of angiotensinogen mRNA in
SHR compared to the PBS control

| Group | Liver | Kidney | Heart |
|---|---|---|---|
| Control oligonucleotide | 5 | 0 | 0 |
| Captopril control | 0 | 41 | 0 |
| ISIS 552668 | 87 | 76 | 89 |

RNA was extracted from kidney tissue for real-time PCR analysis of renin, using primer probe set rRenin2 (forward sequence GCTTTGGACGAATCTTGCTCA, designated herein as SEQ ID NO: 21; reverse sequence TCCCCGCTCCTCCAGG, designated herein as SEQ ID NO: 22; probe sequence AAAATGCCCTCGGTCCGGGAAA, designated herein as SEQ ID NO: 23). The mRNA levels were normalized using the housekeeping gene, GAPDH. As shown in Table 9, Captopril treatment resulted in about three-fold induction of kidney renin expression compared to that with ISIS 552668. Increase in renin in the body is deleterious to the overall health of the animal (Nguyen and Muller, The Biology of the (Pro)Renin Receptor, J Am Soc Nephrol, 2010 January, 21(1):18-23). Hence, the relative lower increase in renin after treatment with ISIS 552668 indicates that it is a safer treatment for hypertensive subjects compared to treatment with Captopril. Results are presented as percent expression of renin, relative to control.

TABLE 9
Percent increase of kidney renin mRNA in SHR

| Group | % |
|---|---|
| PBS | 100 |
| Control oligonucleotide | 162 |
| Captopril control | 1110 |
| ISIS 552668 | 379 |

### Protein Analysis

Angiotensinogen plasma levels were measured by ELISA (IBL International, Toronto, Canada). As shown in Table 10, antisense inhibition of angiotensinogen by ISIS 552668 resulted in a significant reduction of angiotensinogen protein. Results are presented as percent inhibition of angiotensinogen, relative to PBS control.

Immunohistochemical analysis of liver, cardiac and kidney tissues also demonstrated significant reduction of angiotensinogen from these tissues after treatment with ISIS 552668 compared to the PBS control.

TABLE 10
Percent inhibition of angiotensinogen protein
in the plasma of SHR compared to the PBS control

| Group | % |
|---|---|
| Control oligonucleotide | 15 |
| Captopril control | 24 |
| ISIS 552668 | 91 |

### Study 4

Groups of SHR were injected with 25 mg/kg/day, 50 mg/kg/day, 100 mg/kg/day, or 200 mg/kg/day of Captopril administered for 10 weeks. After 10 weeks, ISIS 552668 was administered as a single dose of 50 mg/kg to each of these groups. Two more groups of mice were administered ISIS 552668 at a single dose of 50 mg/kg and 100 mg/kg but were not given Captopril treatment. A control group of SHR was injected with PBS and was given no other treatment. Another control group of SHR was administered a control oligonucleotide (a nucleotide not targeted to angiotensinogen) as a single dose of 50 mg/kg and was given no other treatment.

### Effect on Blood Pressure

Systolic blood pressure (SBP) in the rats was measured before and after dosing with ISIS 552668 by the tail-cuff volume-pressure method (Kent Scientific, Torrington, Conn.). The results are presented in Table 11 and indicate that treatment with the ISIS oligonucleotide was comparable to that with Captopril. Both ISIS 552668 and Captopril reduced blood pressure (BP) levels compared to the PBS control.

TABLE 11
Blood pressure parameters SHRs before and after the addition of
50 mg/kg ISIS 552668 treatment to SHRs given Captopril for 10 weeks

| | | Before ISIS | After ISIS |
|---|---|---|---|
| | | 552668 | 552668 |
| Group | Dose of agent | dosing | dosing |
| PBS | — | 174 | — |
| Control ASO | 50 mg/kg/week | 166 | — |
| Captopril | 25 mg/kg/day | 159 | 153 |
| Captopril | 50 mg/kg/day | 147 | 133 |
| Captopril | 100 mg/kg/day | 137 | 127 |
| Captopril | 200 mg/kg/day | 135 | — |
| ISIS 552668 | 50 mg/kg/week | 146 | — |
| ISIS 552668 | 100 mg/kg/week | 131 | — |

The results indicate that treatment of the rats with ISIS 552668 at a dose of 50 mg/kg/week with Captopril treatment further decreased blood pressure parameters compared to that with Captopril treatment alone. Hence, additive blood pressure lowering was achieved with the combination of angiotensinogen antisense oligonucleotide in animals chronically treated with Captopril, suggesting that improvements in efficacy are possible when antisense oligonucleotides are added to existing RAS inhibitor therapeutics. Such improvement in treatments could be desirable in resistant hypertensive subjects or subjects not achieving their blood pressure goal with existing RAS inhibitors and/or patients that experience ACE escape and/or aldosterone breakthrough

### Example 3

### In Vivo Antisense Inhibition of Angiotensinogen in Dahl/SS Rats

Dahl/Salt Sensititive (Dahl/SS) rats are a model used for diseases associated with high blood pressure (Cowley, A. W. Jr. et al., Physiol. Genomics. 2000. 2: 107-115). The effect of antisense inhibition of angiotensinogen with ISIS 552668 was studied in this model.

### Treatment

The rats were fed a diet with 8% NaCl. A group of female Dahl/SS rats was injected with 40 mg/kg/week of ISIS 552668 administered for 3 weeks. A group of female Dahl/SS rats was injected with 40 mg/kg/week of control oligonucleotides ISIS 141923 administered for 3 weeks. A group of female Dahl/SS rats was injected 150 mg/kg of Captopril administered daily for 3 weeks. A control group of female Dahl/SS rats was injected with phosphate buffered saline (PBS) administered weekly for 3 weeks. Another control group constituted a group of Dahl/Salt Resistant (Dahl/SR) rats injected with phosphate buffered saline (PBS) administered weekly for 3 weeks.

### Effect on Blood Pressure

Systolic blood pressure (SBP), mean arterial pressure (MAP), and diastolic blood pressure (DBP) in the rats was measured by the tail-cuff volume-pressure method (Kent Scientific). The results are presented in Table 12, expressed as the percent decrease in blood pressure compared to the control SHR group. The results indicate that antisense inhibition of angiotensinogen resulted in dose-dependent reductions in all three parameters in the rats. The results also indicate that treatment of the rats with ISIS 552668 decreased blood pressure parameters more than that with Captopril treatment.

TABLE 12
Blood pressure parameters in 3 weeks (mm Hg)

| Group | SBP | MAP | DBP |
|---|---|---|---|
| Dahl/SR control | 157 | 126 | 111 |
| Dahl/SS control | 188 | 147 | 127 |
| Dahl/SS + ISIS | 188 | 147 | 127 |
| 141923 | | | |
| Dahl/SS + ISIS | 146 | 111 | 94 |
| 552668 | | | |
| Dahl/SS + Captopril | 162 | 128 | 111 |

1. A method for inhibiting AGT expression in an animal having or at risk of having a RAS pathway related disease, disorder and/or condition comprising;
(a) selecting the animal suffering from the RAS pathway related disease, disorder or condition, and
(b) administering a compound targeting AGT to the animal,
wherein the compound administered to the animal inhibits AGT expression in the animal having or at risk of having the RAS pathway related disease, disorder and/or condition.

2. A method for treating an animal having, or at risk of having, a RAS pathway related disease, disorder and/or condition comprising
(a) selecting the animal having, or at risk of having, the RAS pathway related disease, disorder or condition, and
(b) administering a therapeutically effective amount of a compound targeting AGT to the animal,
wherein the compound administered to the animal treats the animal having or at risk of having the RAS pathway related disease, disorder and/or condition.

3. A method for treating an animal having or at risk of having RAS dependent organ damage comprising
(a) selecting an animal suffering from a RAS pathway related disease, disorder and/or condition, and
(b) administering a therapeutically effective amount of a compound targeting AGT to the animal,
wherein the compound administered to the animal treats and/or reverses the RAS dependent end organ damage in the animal with the RAS pathway related disease, disorder or condition.

4. The method of any preceding claim, wherein the RAS pathway related disease, disorder or condition is shortened life expectancy, hypertension, chronic kidney disease, stroke, cardiac disease, aneurysms of the blood vessels, peripheral artery disease, organ damage and other RAS pathway related diseases, disorders and/or conditions or symptoms thereof.

5. The method of any preceding claim, wherein the the RAS pathway related disease, disorder or condition is resistant hypertension.

6. The method of claim 4, wherein the RAS pathway related disease, disorder or condition is not hypertension.

7. The method of claim 4, wherein the aneurysms of the blood vessels is aortic aneurysm.

8. The method of claim 4, wherein the organ damage is heart muscle hypertrophy or fibrosis in an organ.

9. The method of claim 8, wherein the organ is heart, liver or kidney.

10. The method of claim 4, wherein the cardiac disease is myocardial infarction, valvular heart disease or heart failure.

11. The method of any preceeding claim, wherein the animal experiences ACE escape and/or aldosterone escape.

12. The method of claim any preceeding claim, wherein AGT has a sequence as shown in any of SEQ ID NOs: 1-16.

13. The method of any preceding claim, wherein the compound is an AGT specific modulator.

14. The method of claim 13, wherein the AGT specific modulator is an antisense oligonucleotide targeting AGT.

15. The method of claim 14, wherein the antisense oligonucleotide is a modified antisense oligonucleotide.

16. The method of claim 14, wherein the antisense oligonucleotide comprises a nucleobase sequence at least 80%, 85%, 90%, 95% or 100% complementary to any of the nucleobase sequences recited in SEQ ID NOs: 1-16.

17. The method of claim 14, wherein the antisense oligonucleotide consists of a single-stranded modified oligonucleotide.

18. The method of claim 14, wherein the antisense oligonucleotide consists of 12 to 30 linked nucleosides.

19. The method of claim 17, wherein the antisense oligonucleotide consists of 20 linked nucleosides.

20. The method of claim 15, wherein the modified antisense oligonucleotide comprises at least one modified internucleoside linkage, at least one modified sugar and/or at least one modified nucleobase.

21. The method of claim 20, wherein the modified internucleoside linkage is a phosphorothioate internucleoside linkage, wherein the modified sugar is a bicyclic sugar or a 2′-O-methoxyethyl and wherein the modified nucleobase is a 5-methylcytosine.

22. The method of claim 15, wherein the modified antisense oligonucleotide comprises:
(a) a gap segment consisting of linked deoxynucleosides;
(b) a 5′ wing segment consisting of linked nucleosides;
(c) a 3′ wing segment consisting of linked nucleosides;
wherein the gap segment is positioned immediately adjacent to and between the 5′ wing segment and the 3′ wing segment and wherein each nucleoside of each wing segment comprises a modified sugar.

23. A method for treating an animal having or at risk for a RAS pathway related disease comprising
(a) selecting the animal having or at risk for a RAS pathway related disease, and
(b) administering to the animal a therapeutically effective amount of a compound comprising a modified oligonucleotide consisting of 12 to 30 linked nucleosides, wherein the modified antisense oligonucleotide is complementary to an AGT nucleic acid as shown in any of SEQ ID NOs: 1-16, and wherein the compound administered to the animal treats the animal having or at risk for the RAS pathway related disease.

24. The method of any preceding claim, wherein the animal is a human.

25. The method of any preceding claim, wherein a marker of the RAS pathway related disease, disorder and/or condition is selected from one or more of shortened life expectancy, hypertension, chronic kidney disease, stroke, myocardial infarction, heart failure, aneurysms of the blood vessels, peripheral artery disease, organ damage and other cardiovascular diseases, disorders and/or conditions or symptoms thereof.

26. The method of any preceding claim, wherein the compound is parenterally administered.

27. The method of claim 26, wherein the parenteral administration is any of subcutaneous or intravenous administration.

28. The method of any preceding claim, wherein the compound is administered at most once daily, at most once a week, at most once every two weeks or at most once a month.

29. The method of any preceding claim, further comprising a second agent.

30. The method of claim 29, wherein the second agent is administered concomitantly or sequentially with the compound.

31. The method of claim 29, wherein the second agent is an anti-hypertensive drug, a procedure, an anti-fibrotic drug, a diet change and/or lifestyle change.

32. The method of claim 31, wherein the anti-hypertensive drug is selected from any of a RAS inhibitor, diuretic, calcium channel blocker, adrenergic receptor antagonist, adrenergic agonist and vasodilator.

33. The method of any preceding claim, wherein the compound is a salt form.

34. The method of any preceding claim, further comprising a pharmaceutically acceptable carrier or diluent.

35. The method of any preceding claim, wherein the compound targeting AGT inhibits the RAS pathway by at least 70%, 75%, 80%, 85%, 90%, 95% or 100%.

36. Use of a compound targeted to AGT for treating a RAS pathway related disease, disorder and/or condition.

37. The use of claim 36, wherein the compound is an AGT specific inhibitor.

38. The use of claim 37, wherein the AGT specific inhibitor is a modified antisense oligonucleotide.

39. The use of claims 36-38, wherein AGT has a sequence as shown in any of SEQ ID NOs: 1-16.


SEQUENCE LISTING (23 sequences)

SEQ ID NO: 1 - DNA, 2587 bp, Homo sapiens
  CDS (509)..(1966)
atcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcatcgtgacccggccgggggaagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaaaaa

SEQ ID NO: 2 - DNA, 1670 bp, Homo sapiens
agggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttccc

SEQ ID NO: 3 - DNA, 1399 bp, Homo sapiens
agaagctgccgttgttctgggtactacagcagaagggtatgcggaagcgagcaccccagtctgagatggctcctgccggtgtgagcctgaggaccaccatcctctgcctcctggcctgggctggcctggctgcaggtgaccgggtgtacatacaccccttccacctcgtcatccacaatgagagtacctgtgagcagctggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactgtctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcatttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctg

SEQ ID NO: 4 - DNA, 1584 bp, Homo sapiens
  CDS (39)..(1439)
agaagctgccgttgttctgggtactacagcagaagggtatgcggaagcgagcaccccagtctgagatggctcctgccggtgtgagcctgagggccaccatcctctgcctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgacgggagccagtgtggacagcaccctggctttcaacacctacgtccacttccaagggaggatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactgtctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgag

SEQ ID NO: 5 - DNA, 1691 bp, Homo sapiens
  CDS (40)..(1251)
aagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactgtctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacggcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcgcaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgta

SEQ ID NO: 6 - DNA, 16101 bp, Homo sapiens
catcagaaagatccaccctcatgattcaattacctcccactgggtccctcccatgacacatgggaattatgggagctacaattggagatttgggtggggacacagccaaaccatatcagatggcttatttggtttctatgtagaacctctgcttttcattcaacagtcttcatttagccacagataagctctgtccctaacttccactgatggaatgtacacataagaaacttccactgatggaatgaacacagaaggtgcctactgggaagaaaactggcctgaatctgagctgggtcaaatgtctgcagtcagtttgaatggctgctccttatgggaataatttacattctcaataaaattctctagcaattttctgattgattttaatgagctttaaagccttacgtagaagatcccccagctgatagtcagccttgggcatggattaagggcttttaaccaatcttgcaacaagtttaagcagatattctttattgggtccaatctaaccaaaattattttcttatgttctccccagtaacgtgtcattattaagagaagtttggcttgcttagaggccaaatttagagggtcctgaaattttattttcttttacaccactttccagcatgttacctgatcagttgtttattatctttgctgttgaatggagtgatcattccaagggcccgaggcaggaggcccaggcacagtggaaactctcccaaagaccaggatctttgttttgttccctgacatatgctgagcaccaggaatagtgagtgaatgaaacaaattgtgaggctttaaagagccgaaatatttaaacactgggcacaaggttgttgcttaatcagtgctagatccttacctcccccttgtgtccaggtggacttgttactgcagttaaaccacttgctgatcctcaaacaactagttagtggcacagccaggcctaggaccccagtctctactgttccaactaacccattcgcaggcaggagcactttgaatggtctcttattttaaaaaaattaaattaaaattgtctatttatttagagacagagtcttactctgtagcccaggctcgagtgcagtggtgcaatcatagctcactgtaacctccatctcctggcctcaaaaagtgtttgaattacagatgcgaggcactgtacctggcccgaatgttctgttcagacaaagccacctctaagtcgctgtggggccccagacaagtgatttttgaggagtccctatctataggaacaaagtaattaaaaaaatgtatttcagaatttacaggcccatgtgagatatgatttttttaaatgaagatttagagtaatgggtaaaaaagaggtatttgtgtgtttgttgattgttcagtcagtgaatgtacagcttctgcctcatatccaggcaccatctcttcctgctctttgttgttaaatgttccattcctgggtaatttcatgtctgccatcgtggatatgccgtggctccttgaacctgcttgtgttgaagcaggatcttccttcctgtcccttcagtgccctaataccatgtatttaaggctggacacatcaccactcccaacctgcctcacccactgcgtcacttgtgatcactggcttctggcgactctcaccaaggtctctgtcatgccctgttataatgactacaaaagcaagtcttacctataggaaaataagaattataacccttttactggtcatgtgaaacttaccatttgcaatttgtacagcataaacacagaacagcacatctttcaatgcctgcatcctgaaggcattttgtttgtgtctttcaatctggctgtgctattgttggtgtttaacagtctccccagctacactggaaacttccagaaggcacttttcacttgcttgtgtgttttccccagtgtctattagaggcctttgcacagggtaggctctttggagcagctgaaggtcacacatcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcatcgtgacccggccgggggaagaagctgccgttgttctgggtactacagcagaaggtaagccgggggccccctcagctccttctcggtcttgtctctctcagatgtaactgagctgtgggctaggaggaaaaggccgggaggaggcacggtgatgactgaaaaacctctcccctctcataagaccagtcatccggacgcgggctttcccccactcggtgcccacctggggtcttacaggaggagctgctcctcctcagcaataggacaagatggtcaggtcttcctgcttccgctgagaaaagttagggtcctcaggaacggagcagactggtacaggaacagagtcatcatggccaagagtccaccgggtcctcttgccatcaggaggaatagcagggcttgtgcaggaattggggctggagggaagggccgggctcggtcagtctccagctgggatccccagagtggtcaccctacccctccctcgagacagactgcctgactgtgtgtcatcaggctggtcaccatctccctgaacctcgatttgctcacctataaaatggaactaataacgatgcctgggctccctgtctcaggggctctggtatagctgaagagaactaatataacatgaaagtgctttctaagctttgggataagctaaaaggcagattccaattttattcgagggcagcgtagattggtgcttcagctcgtggatgacagagtcagggggcctggttctgagtcctagttctgtctcttcccagctgtgtgacgttgaacaagtcactggacctctctgttcctctgcaaaacagcatgaaccaattcattaactacttctccaggatgcagtaggtcccagggactatcctaggaatgtgggctgtattagtaaacacaacagcgggaaccctgttccggggctcacattcacatcagagcaaacagacaaagacgctggacagaataagtgcataactacatggtacagagggttataaggagggaaaaggggagctggatgagagagttgagagtgcccggtgtggtggggaaagctgcagggtgaaatactgcatcagggaaacctcagggaaggtgaggactatggtgaggtcagaggggttgatatgagaacagtgccctgcaaatggcaggcaccacaggagcatgagccgtcatcttcacctttagcattcagcccgggagaagtagggagacatagaaggggcaggtgctggccaagaggcaggggcaggagaggagaaggcggaggggcactcagggcgagggtgtcaggcccgccaccccagagcaccattactcccaggacgcggctgcgtgcagacctggaaccagcctagggagcagccgcagatcacaactgagaacaaacgacagtctctgcctcaaaaatggcccatggaattgcgtctctggagacgctgcctgagcaggagcagcacagtgagcgggctgcatcgaccagcgccatccaaaccccgaacagttggcgcttgtcaggcaggacttcccagcagtcggttcccacaggtttcccctgttgacctgatttgatgtgactgtctagattaggtgtgaactggtggcttaggcttctctgcacagaaaggcctgcaagcagcagagagagttttctgttccatttttccatgtcatgtggctcttcctgagaacagcggatggagtcaaatgcatggggagtggggtgagatggtagctgaggtcagaatttggcatttgaatgactgaagcagaacaaaacacaccaggtacttcagcagctgcaccgtgttgagggcaggtgctggttacgggtctgggtgagggaagccagctgccaatgtaagaagaatgactgggtatgcttagatgaagcagaaaaatctaggcatcaaggtggccttgagtcagtgatgacacgctacagctccaaggaagcctggcctagccctggggggacagaaaaggccaagaagtgacgatattgcagtacacccccctccacaagaaatgagtgagatgtggtacaaaatgttagaattgaatgaatcaatagaataaacgttcatcccttcaatcaagaagagtcagatgaaatgaattagcagggccagcccaagaacctcttctgggggtctcagggtagctttcatttgtagcagctgaggctgaagcccagctgcaaggcctttgagagaacgtggtgctggacccgtgtctagggcaggggttctaaaccctgcttacatatcagagtcacctgagaattttctatttttttttttttttttttatacgtggtcccagcacagactaaggaatccaactatcattgggcaagccatgctaggtatgcatgcctttggggctctgcaggggatagcgctatgcagggatggttgagagctggttttggggttgagacacgtgggaaatacttggactttgggctgagcctgtggtgctcaatcccggctgcatgttgggaccacagggagatgacaaaaccatccccagccctcaccctagggccctcgaatgagcatctcaggggtctaggaggcctccacaaagacctactgattggcacacacttgtttctctaggaagagaacttacagctgcaggcaggagcatgtcttaatctgcttgggctgccataagtaccacagactgggagggtttaacaacagaaatgtgttatctcacagttctggaagctagaagcctgggagccagccatcagcagagttggtttcctctgggtcctctatccttggcttgtagatggccgtcttctctctgtgtccccacatggtcttccctctgtgtccccacatggtcttccctctgtgtgtgtccatgtcctcatctcctcttctcataaggacacaggtcatattagatcagggctcaccctcatggcctcattttaacttaatcatctctttaaagatcctgtctccaaataatggtcacattctgaggtcctggggttgaggacttcaacacgggcattatggccgttgggggaggtaggacataattcagctgatattggtgcattttgcacttggatcatgtagatattttccatggagctttgaatccatttcttcttttttttgtagacatgaatggatttattctgggctaaatggtgacagggaatattgagacaatgaaagatctggttagatggcacttaaaggtcagttaataaccacctttcaccctttgcaaaatgatatttcagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaaggtaaggcaaacctctctgctggctctggccctaggacttagtatccaatgtgtagctgagatcagccagtcaggccttggagatgggcagggggcagccctgcggacatacctggtgaccacccttgagaagtggggaagtggctgctccgctgggtccctggatgggccgtccacctcctggacctgctgccctactatgtgcacgactatacaacatcctttttcttacatcatttaatccccttatgatgtggtgaagaggtatttgtgcctttgtttaccagtgaagaaatagagactcggagaaacaaagtgccttgctcaagatggcacagccaccagtgggggtcctgggattgaaacccacatctcctggccccacagcccagttctacactcagaagggtcaggttcatatctcttgagaaggtcaggaactggggtccctggcccatgcagaaataagcaattggcttgcttaaatccctttcatgttaggaggggcattactgaaaaccctctactacaaagattgttgatttttttttttttttttattgagacagggtcttgttctgtcacccaggctgcagtgtagtggtgccatcattgctcactgtagccttgaactcctggcctcaagcgatcctcccacctctgccttccaaagtgttgggattaaaggtgtgagccactgcacccagccacagattgcttaaagcattcatttaacaaatacttgttgaggatttgctacttgtaagactttaagcctggcatctcagaggaggccagaggagggctgtataggccctgcctccaggcttttaaaggtcaatgggcaaatgcctaggatttggagctgcagggaaacgtgctccacaaggtaactcagggaagcctcggggctctcagaggacagaggtcactggggagcggagagcaggccttgcctggcagtgagggcaacagggctggtgaagctaggagcaagcatgatgagcccagcctgcagagtttggggcaaggaacgaggatggggcggttggcttggcatgagtgttgaaccagaaaatgggcctggggagggcagagctggagacactttgaacgccatgcttggtaggtgtgggaatggggacgcgttctgttcagaggtcatcccggaagcctgccgtgtgcagactggaggcagggaggattgtttgaaggttacgcaagagtccaggcacacagtcacgggaacacgtgctcagggagcagctcggcaaatccatgggtggggtggggctgaggggtgtgtctaagagacactgaggaggctctgtcaagatgttaacctcgtgagggacagagagccaggcgggaggtgaaagacaagactgtggagaaagaggttcagtggcgcatagtgatttttcttaccacaacaacctccttgaggtctttcccttcgggttcagggagaggtgatagatggggggattgctcagccctggcactgactggtcacaggggcagaggccagcccgagggttgcccggttgagggtggcagcacactgtgcagggcagagcagggacacatggacttagcctgctgtccctaggagaagtgctgggaggagcgctcactgagaaggagggtcctgcagaaggcaaaggcaagaaagccagtggcatctgaaatgggtctcccttcgaaagagagcacatccacctgacccagaccgcagagccaggccaggaggaagaggaggaagaataaaaaagccaaccacatcgggactcaaaggaagcccaggatcctcgccggcctccaccgcatgctgccctgaccctgccccacttcctaactttgctggcctcagtttccgtcaaaggaggcagccacttcctgcccacatggtctgtccagtgaggagatcgggggctgtctcgggacctctaggtttccctttagcaatgatgttctatttacatgacctcagcaggcagctagatgtgtcccactagagaggacctgaggatctggggcctgatgggctccagggtaccgtctgcccagtgcttgctgtgctcctgagcatggggcgctggccctggtggtttccatgacaccaggtcctgacttgacctcgacagatttacctagcctccggatgagaatggtgagctgtgcatgtcagacgagcagagggaagacggcagccactctcatgtcaaatcccagcgtcttttgggaggcagcttcccttttttagtttagtttgttggaagaaaagaattgtccctttcccccctctaaactaaaagccttgccagcccaggtgggcagcaccgaggtccctgcagggaacgtgcaaggggaaccctgcagtttcccgctcacatgcccttccgagactgagtgctccgaggactgaggacgagaaatatgccaggtctgccactgccttcttacgagacccggacccaggggaggcacagccatgcccagctcctgcctgccagttctgtcctcccagctgccctactttcatgctgggacctccaattcagtacaaagggagacctcactgtttctgaaccatctctactcagactcccaagtgccacgtgcccaggggactgttctgtgacaaacttatacacaacttcaccctattctcctaagaacaaccgcagaataggcctttcaggatgagtgggaggacagccgagggcagggatgtgctagtgtaaggtcgaggcagagggtgggctgctgtcatggaaagaccccaggtaactgcgtcacacacaaatttgtgtccttctcccacaacgggctctcccgagttctctgtcatctgcacggccctgtgagcaggaggggaaacagagggctcacccctgcccccaaggcccagtgtgcaaatccattcatcacaacgaggttgtgtgagtctccccagtagcaagggctgctgaggaatggagccctcgtttccggggcctgcgtggcccactctgtattctatgactgtgatgggggagggtgggggccacaggacagctggtgggctctgccatggctggggctagacatggattaaaaagtgagtatgagcaggggcctctaggagtggtgggatagtgcggtggtggccacatgtcattctacgtgcgtccaaacctacagaatgtaaaacaccaggagggagactcaaagaaaactatcaactttgagtgctgaggacgtgtcagtgtaggttcgtcagttgcaacaaatgggccacgctggtgtgagatgttgatcacgggggaggctgtgtagtgggggacaagagttatatgggaactttctgtactttctgctcgattttgctgtgaacctaaagtcactctaaaaaataacatctcttaaattttttaaaaagtgagtgtgtcaaaccacagcctttgggtcaggacagttctaggtttgagttgacctggcaggtaccagtggcttatgtcccttaaggtgacagatgcaaaacccccggtttggtgcctggcatgttgtgtgtcttgcaggtggcggttagggctgcctcagtgaactcaaatggctgcattttacaggagaaatatttgagccacacttgcggtcctgtggccaggagaatgcagagtggcctggggggggccaaggaaggaggctgaggcagggcgaggggcaggatctgggcctttggtgtctgccagccctcattcctgcccctgtcttgggtgactcttccctccctgtctcctgtctggatttcagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggtaggagcctcccggtctcccctggaatgtgggagccacactgtcctgcccaggctgggggcggggtggggagtagacacacctgagctgagccttgggtgcagagcagggcagggccgcggtggcacggggctgggcaggcggcctgtgtgtctgtctaccagtcctctatccagccagcacccagctctccagttagtgtctgtctttcaagtgcaggcaaggtaaaggaggagaggaagaatgctttttctacacttacacttgcctggtagttttggagggggagaaaacattgcaatccgccctctgagagaggaccattttggtcccacacctgacacacagcacacctgtgacatccaagagcttcttggaactgacttgccaggagggttcggacttcgcgtgagcgggggtggggccttctcagggagcgtcccttgactccagaacgcccttgctggcggctggcggctgggtggggataggtgttgttagctcctctttcctgctgcaattcctttccacagagccctggactcaaactacacatcaccccagatcatcgaggcctggaaatctgctcccagaggcaggcattgagtgacacgatggcttgacatcaactctgggtgttttttatgttttaaaaattgtgatggtaaaatatacgtaacaaaatttgccatcgtaaccattttcgagtgcacagttcagtggtactaggcccattcacactgttgtgcagccatcacccccgtccatctccatttatcttctcaacttcccaaactgaagctctgtcctgctgaaacactaactctccatttccccttccccttggccccggcaaccaccacgatgtcctcgaggttcacccatgttgtagcacatgtcagaatgtccttccttttgaaggctgaataatattccattgcatgtggttaccaccttttgtgtatccactcatccatcgatggacacgtgggttgcttccacctttgagctgctgtgaatagtgcagtgtaccctgtaaacatgggtgtactgtcagctcttataagtgcttgatacatcactggaaatgtccatgggctctgaaggatgccaaaagatggaagaggctctatacgaagatcaatcgagttgacatagcaacgtgtccagcacgaggttgacactgtaccctcctgcctctctccttttcatgggtgtcatgtcatcaagaacactgctgtggcagtagtaagacacagtgcattatttcagagaatagcatttaaaaattacccaagtaacacaccttcaatgcagccaacctaaaaacagaatgcaccaaaggacaaccattcctaggtcctcatcggtaaatcttctatgtccctcacatagtattgcaaatgacatgaaggatttttattgtaggttttgctgaaattttccccaagggggaggatgacttagttgggtgatggggggagcaaacatccctgtcgtcagggttgggtgcaaggagcataagcctgcctggcctctgggagagccctcactgtgtggcctggagccttcctaactgtgcatcatctccccaggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtatgtgtgagcctgtgtctgtgcctgacctgggttccaagtgtgcacagggtgggaggcatggatgtaagggacacagaggaggctatgggtggggccagcagggcaagagggagcggagagtagggccaaaggtgggagagaagtagccagagcattctggggccttccaggtgcagagcagcaaatccctccccatccctgctgtgcctcctcctgctaggtgtgtgttccatggtcctgcttggccttgccttgcctcagggtcctccagggttcctatagtggagttgaaaccgggatgaagacagcaagcacccctggacctggtgccctgggcccagccccttcttcagggaaatgctgagcagcagacagaatgtccccctgccatgtggcaccatgcacatctgcagctaccaaggatgtgccttgatgttctgggccctgtgctcagtgctggggagaaagtgggagttcttacgggggccagcgggaagagccctctgtgctaagttagctaagccctggcactggtgggccatggccaagggagccaggaattctgcctgggacatcagggcagaatgtgaagatgggaggatgtaaggggtgtgttagggaggagccggcatgtgagtttggccattgtggccaattaacggtcatctacacacagacacacccttgcctacactgaggggcaggcatacactgtgcatcctcctggcaggctggaaaatgtccccctccaggacagtgcacagcacagaggtcctgagcccaccccggccctctagccctcagcaccctgggtcacccagtgcgccctcagaatgatcctgatgtctgctgctttgcaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaattttatctttcatttagcagccaaacagatgtatacaattcagcagatagactgtgcaaacgaaagtgctttcctggactttggatggaatttccatgggaggtctgagccagtacttagcagtcctttgaagttttaggtgatgcttttctctggacacttccattggtaagcagtggtggccatctgtgtgatggacagggggcgggaagagggtgacagggaaggccccataccccatgtggcacctgggaaaggaaccaggcagatgggacttcttccgtcctggtgacacagggccagactgctgctggtattgtgccccgggagtggaaggtagagaaataaatcttcacaaataaatatttgcaattttcccccatctgttgagtgcctctgcctgctcctcctcgatgggattaggcccacagttcggaatcttggggagagccaaggaagcggtaggcacccagtaggcccacggccgtcggctgatagcaatggtgatgctgtcctacctacttgtgtaaggcattcgatcttcctcccttccatacatattgaaataaataagccgcgcaatgtgttagctattgatcagaactaaagtgaagtcagccacggggattacaaatctcggcttctcccctcatgttcctgagagtcttcccctggttttgaacacatctccctagctcgatgtcaaggtgagggattctgtcggcaacagcagtgcccttagttgcttcgtcgtaactccccgtcaccggttttattcagttaccttccagtcccactctcagagcttcctggcttgttctgctctcaaagcgggtagagctggcacacatggactctccgaaacggctgcaagatgccaagtttctcggaagaactggaagcacagagaccagaagtgccttaaggtctcgctattcagtgtggcgcttagaccggcagtggcggcagctgccctgggagcttgttagaatgtggcttctcacgcccctcctggacctacagagtcagaatctgcagttttacaggaggtccaggcttggaagttgctcgtagagacctgagacagcgcagccacgtgctggaaacaaagcatttaagtttgtgactttattttaaaaggcagcaggcagtcgacaaaccaatttcttctacttagaggcggcttcggcttctggaagtcgctaggagtataaagttgccaaccagcgctgttctcccgctgttttctgtgcacttataaatgggaagttaggtcaggatagatctctcagctattacaaggatacaaaatacgaacattctacaagttacttaacacacacacacacacacacacacacacacacacacacaaaattaattccacaggtcagtttctctgaaacattttttcactaaattctaagtcttcctggagttgcaagtgcctatctcctagacaaggcaattactcaccaactaaaatcactgtcaatctgagatttcggctgggcatgagaccatggtcaggggatgctttgaacagcctctgaggaaattagtgagtttgaaaaatggaaagatttttattactcacttggcagtaaaacctgatggggacagacgtcaggctgtttaagatcctcagaagaaaaagttgatagtgtgaatattcctaaatttgccacacgaagatgtacatgtgattataaggtgctgttgcagaagcccctgggggtgttatgggatatacactatatgggccactttaccttcctaaaatctgaaaaacttcaactactgaaacatggactgaaggttttgaatagtggatggtgaatttgaataccatcccgtgtgatttttttttctagcagactttagttttttagagcagttttaagcccacaccaaaactgagaggaagatacagcaatttctcatataccccctactaccttccagtctcccccattattgacatcccccacccagagtggtccatttcttacaacccacgaacctacattgacacatcattattactc

SEQ ID NO: 7 - DNA, 1856 bp, Homo sapiens
  CDS (48)..(1496)
gatagctgtgcttgtctaggttggcgctgaaggatacacagaagcaaatgcacagatcggagatgactcccacgggggcaggcctgaaggccaccatcttctgcatcttgacctgggtcagcctgacggctggggaccgcgtatacatccaccccttccatctcctttaccacaacaagagcacctgcgcccagctggagaaccccagtgtggagacactcccagagtcaacgttcgagcctgtgcccattcaggccaagacctcccctgtgaatgagaagaccctgcatgatcagctcgtgctggccgccgagaagctagaggatgaggaccggaagcgggctgcccaggtcgcaatgatcgccaacttcgtgggcttccgcatgtacaagatgctgaatgaggcaggaagtggggccagtggggccatcctctcaccaccagctctctttggcaccctggtctctttctaccttggatccttagatcccacggccagccagctgcagacgctgctggatgtccctgtgaaggagggagactgcacctcccgactagatggacacaaggtcctcgctgccctgcgggccattcagggcttgctggtcacccagggtgggagcagcagccagacacccctgctacagtccattgtggtggggctcttcactgctccaggctttcgtctaaagcactcatttgttcagagcctggctctctttacccctgccctcttcccacgctctctggatttatccactgacccagttcttgccactgagaaaatcaacaggttcataaaggctgtgacagggtggaagatgaacttgccactggagggggtcagtacagacagcaccctacttttcaacacctacgttcacttccaaggaacgatgagaggtttctctcagctgcctggagtccatgaattctgggtggacaacagcatctcggtgtctgtgcccatgatctccggcactggcaacttccagcactggagtgacacccagaacaacttctccgtgacgtgcgtgcccctaggtgagagagccaccctgctgctcatccagccccactgcacctcagatctcgacagggtggaggccctcatcttccggaacgacctcctgacttggatagagaacccgcctcctcgggccatccgcctgactctgccccagctggaaatccgaggatcctacaatctgcaggacctgctggctgaggacaagctgcccacccttttgggtgcggaggcaaatctgaacaacattggtgacaccaacccccgagtgggagaggttctcaatagcatcctcctcgaactcaaagcaggagaggaggaacagccgaccacgtctgtccagcagcctggctcaccggaggcactggatgtgaccctgagcagccccttcctgttcgccatctacgagcaggactcaggcacgctgcactttctgggcagagtgaataacccccagagtgtggtgtgaggccttgtgcctagccatggagacaaggccggtgtcggagaaccgttctgggcaaaactcagtgctgtcacccctggctccccatcacgccttgtagcgcggcagaggccgtctccttggagactgcgctgaccgagaataaatgatgagcagcagagcctcctgggatgtgggtttgtttggatactggggtgacagccagaagctggcactctgcacaggactgccactctggaagaaatttggaccaaaaaactgtttgtgacaccaaaaagcacccccccttttttttatttgaggacagaaattgggttttaacattaaaatgcacattatccccttaaaaaaaaaaaaaaaaaa

SEQ ID NO: 8 - DNA, 358 bp, Mus musculus
agccagacacccctgctacagtccattgtggtggggctcttcactgctccaggctttcgtctaaagcactcatttgttcagagcctggctctctttacccctgccctcttcccacgctctctggatttatccactgacccagttcttgccactgagaaaatcaacaggttcataaaggctgtgacagggtggaagatgaacttgccactggagggggtcagtacagacagcaccctacttttcaacacctacgttcacttccaaggaacgatgagaggttctctcagctgcctggagtccatgaattctgggtggacaacagcatctcggagaaccgttctgggcaaaactcagtgct

SEQ ID NO: 9 - DNA, 457 bp, Mus musculus
cttgtctaggttggcgctgaaggaacacagaagcaaatgcacagatcggagatgactcccacgggggcaggcctgaaggccaccatcttctgcatcttgacctgggtcagcctgacggctggggaccgcgtatacatccacccagttcttgccactgagaaaatcaacaggttcataaaggctgtgacagggtggaagatgaacttgccactggagggggtcagtacagacagcaccctacttttcaacacctacgttcacttccaaggaacgatgagaggtttctctcagctgcctggagtccatgaattctgggtgggcaacagcatctcggtgtctgtgcccatgatctccggcactggcaacttccagcactggagtgacacccagaacaacttctccgtgacgtgcgtgcccctaggtgagagagccaccctgctgctcatccagccccact

SEQ ID NO: 10 - DNA, 830 bp, Mus musculus
cggacgcgtggggtctaggttggcgctgaaggatacacagaagcaaatgcacagatcggagatgactcccacgggggcaggcctgaaggccaccatcttctgcatcttgacctgggtcagcctgacggctggggaccgcgtatacatccaccccttccatctcctttaccacaacaagagcacctgcgcccagctggagaaccccagtgtggagacactcccagagtcaacgttcgagcctgtgcccattcaggccaagacctcccctgtgaatgagaagaccctgcagacgctgctggatgtccctgtgaaggagggagactgcacctcccgactagatggacacaaggtcctcgctgccctgcgggccattcagggcttgctggtcacccagggtgggagcagcagccagacacccctgctacagtccattgtggtggggctcttcactgctccaggctttcgtctaaagcactcatttgttcagagcctggctctctttacccctgccctcttcccacgctctctggatttatccactgacccagttcttgccactgagaaaatcaacaggttcataaacggctgtgacagggtggaagatgaacttgccactggagggggtcagtacagacagcaccctacttttcaacacctacgttcacttccaaggaacgatgacgaggtttctctcagctgcctggagtccatgaattctgggtggacaacagcatctcggtgtctgtgcgcatgatctccggcactggcaacttccagcactggagtgacaccagaacaacttctcgtgaagtgcgtgccctaggtgagac

SEQ ID NO: 11 - DNA, 633 bp, Mus musculus
tcagtactgtgcgtgccctcaggtagcagagccaccttgctgtcattcagcctcatctgcacatcagatcttcacagggtgcaggccctcatcttccgaaacgacctcctgacttggatagagaacccgcctcctcgggccatccgcctgactctgccccagctggaaatccgaggatcctacaatctgcaggacctgctggctgaggacaagctgcccacccttttgggtgcggaggcaaatctgaacaacattggtgacaccaacccccgagtgggagaggttctcaatagcatcctcctcgaactcaaagcaggagaggaggaacagccgaccacgtctgtccagcagcctggctcaccggaggcactggatgtgaccctgagcagccccttcctgttcgccatctacgagcaggactcaggcacgctgcactttctgggcagagtgaataacccccagagtgtggtgtgaggccttgtgcctagccatggagacaaggccggtgtcggagaaccgttctgggcaaaactcagtgctgtcacccctggctccccatcacgccttgtagcgcggcagaggccgtctccttggagactgcgctgaccgagaataaatgatgagcagcagaaa

SEQ ID NO: 12 - DNA, 16201 bp, Mus musculus
ccgtcactctacagccctgcctcctacagctggtctcccactgagatctctggtagttattttatgtctgtgcccagagaaggttgctcttcagagcccatctcaaacaccatcaagtcctgcatggtgcatctgtcttctaagccagcgactgctctgcccactctctctttccacctctactctcccatgccacttttaggtgaaggcaggttgtgcctgtgtccacatggccaagagattgcttgaagttgtggatgcgatgcaagctagtcgccacccatggtagcctgcaggtgggactgtctgccaggcctactcctgctgtcttctcagatcccacccttccctggtgtccaccagtctcatcccttaaactttcacaaccattctccaccctcaagggaggcgctccaaaacctccaacctccaaaactgctgctttccgtccatgagcccagaacattttgtttcaatatggctctaccacagtcagaatttcccagcctaatcaggtgtgctagaagttccggaaggcgcttcctactggtgatgtgtgtcctcagcccactggaagcccctgtacagaacaggcaactaacgtaactacaaatgtccaaggtcgtccaccctgagagcagctggtgacgtgaggttcagctcctgcaagtgagcccccttcctggcatgcccagagaggcttacgagtgcatcacgagggggctttcatcccaaggtctgcatggctggcttcaggttgtcacaacccactcaatcctgtgactgtggtcctggctccagggaactggggtaaatgtgtaacccaaggccagcctatttttgcatgaggctcatctgccagtagggcttcctggaaggggcccagaggaacatcaccctggccctgatccatcttggtcaagcctggattctcatggttccctgatctgggtcctcccccagcctctgtacagagtagcccaggaatagatccatctccaccccttgagtataaataagcctgcttggctcaccaggggatagctgtgcttgtctaggttggcgctgaaggtaagcagagcactcttgtcttggttctttcatgggatctggccaggcaggagctgagctgtgggtgggaagaggaggccgggactgggcagagacactgggaagggctggatggcattttttctggagtgagaagtctgtggtagcaggcgcttcaggagatgtgagcactgactgcttctcctattccgaaaggctagatgtccaggtaaactgtcccctgtgggtcaccaaactgggctcactgggaactgatggtctttcattaggggatggccaggtccttttgcctcctgtgagtccagatgggtcctcaaaagcagagagactctcacagggtcagcactagggtggccaagtcccctggacctcctccagcctgaagggagaacaaggcttcattgttcagactgcaagaagcaggaagggacaagtcacgtgacatctctatccaggctcaatgctgtggtctgtgtcttgtggctcctcaccatgtcctgtagggcctcaacttacccctctgtgaacaggcttcatcacagttctgaggttctctagggctggggataagttaggagcatccagtaacatggaagtactttgaaaagttagctccttctttattcttagtgtggtgtgtgtgtgtgtgccgtgttgtgtatgtgcatattatttatttatttacttctggtttctgagacagggtttctctgttgtagtctttgctgtcctagaactcactctgtaaagcgtgcaggcctcagattcagagatatgtctgcccctttttttttttttttttttggaagacagggtcttactgtgtagctctggttgtcctagaactcactaaattctgtctgcctcaacctcttgattgtatcaccatacccagcccaaggtatatatgtatttatgtgaatgtgacatatgtgtgtatgacgtgtgtatttatgtacatatgtatgtatgtatgtatgtatgtatgtatgtatgtatgtatgtatgtgtatatatgaagtgtaggggaggagcatggagacataggagccatgaacacagatagagactagaggaggataccaggtgtcctgccagctactgtcctccttagtcccttgagacagggtcccttcctgaagctggggctagactggcagccaggaagccctggagatcatcctgtctcctcgctccacctctctggagttaggggagtatgtggttgtgactggtctttcacaaggggtcaggggacgagaacccggattttcatatttatgcaacaacgtgttcttgcgtgctgaacctgccctaggctcctcagttccttatttcttttaagaggcaatatgtgtcatgtctagatgccactcatggattcaagtgttagggctgagtcccagctgtgtgacctggtgcaggtcacaagacttgtctgttcctgtacaaatggcactgatgattctacagctatccctggatgaccttaggaaggtgggctaacctgtggcactcaggtacctgtgggaggcagaccagtggggcacatagtaaatacatcgatacaggatatagggtgcttctggtggctatgaggtacagaggaggtgagagagaggtgtggggagcctgaggtagactttgagtggtgaagatgaactattgacagaagacagcactggaatgcaccctgataatgacaggacaggcaacatagccctgagaagccacaagacagggacacagagaggcagctggtggggagggaatagaagtgtgtgtgtgggggtgagatctcctatgtcctcaagatttgctgcaatcacacttagggtgaacttgactgacagcagcttacagctaagaacaaacagtcacttgtgcctaaggacagtagtgacgcgtttcctgggacagcagccatggcactaccttctggacagaggccgttcataggaacagcagtctaggaaacattgtgttcacccccacagctcatccagtggcctgggcacctgaggcaggtcctgaccctactgtgttggattataaagcaaggaaaggccagtgagtacaggcagagctctctactctgctttcccaaacctctaaactttctttaagaatgatgggtgaagccagatgtgtgtgtcaaaaggatgaagcactagacagtcagaagccaaaggttctcagaccagtgggatgaccagagcagagcaaagagcaccttgtctgtctgtggaggatattctatgtgcagcctcggcaagcacgggaatatgggagtccagggtgaccttggagctttgcaactccatagtgacattctgagatcattaacttatgaagagaaaggtttgtcttggctcatggtactggaattgcaggtcaaattcaggtagactgttgctcgggacttctggggtgttggctgacaatggcaatgacatctggtaaaacgagctgcttccaatgtagctaggaagcaaaagcaacgaagaaggaaagacacaggtcctgcgacccctcttaatggcatgcccccagtggccttgggcccccactaggacctacaccttaaatgtctgcagcaccttcaacagaacaatacggacattaagatcctcacctagggaccttgcaggtggtttggtggagggttgtacatggaaaggtgaagcagcagaaaggaagggtcaaggtgacctcagtcagggttgaggtgccaaggctcacagtggtggccacagtgaggtaaggggttggaagccacatgccaggctggttcctatagaagcaagcgcacatggtctgcttggtcagaactgaaggatggatagaactcaccttcccacactaggaccagaggaataagaggtaggctttgcagagtcagctcaggagccttcctctgggatccaaggctaagtctatgtactgtgggtggtatctgaggctgggaaaggctatccagctcaatggaccaccccatcccctggtccatgactggccagagccccaagcccttcctgtacctcatagccatctgggtgaatcaattccttgccttaccatagaccactgaaaccaaccgtgggtgagcaggctgggcagatgaaccaattatagagtcttctttagctgctgtgtgggatccagcctgcctggagcatatctgaaggacctgggcttgggcaaggcctttggctctcaattatggctgcaggctaggatctcatggacactaaaatcaaccctagggcacacccacgtgggtacaaaggctccacaaaacccagtgggccatccacatgtcctccaggaataaactcttagcctttgtcacttgtatgcctggttctaccaggactgtgccacagcctagtatgatagataacaagagaacctccagaaacccaaagcaaggcatggtcagagccaagttctcctggggcctctctctgcctttgtcttgcaaattccctcctctccctgtgactctaatagatttctcttcctgtaccctgatatcctcgtgtgtgtgtgtgtgtgtgtgtgtgtgtgtgtgtgtgtgtgtgtgtgcgcatatgtgtgttctcatgtgtatagtgactggatgacaacctttcatgtcgtgtcaaaatggtgttcatctcctttgagacaggctagagagaaccctagggagtcaagccccaggaatctacctgtctccagtgcctgagctctgaggttacatgcatatgctattgcacctagtatttttttaagatttatttatttatttatttacttatttatttattatataagtgcactgtagctatcttcagacacaccagaagagggcatcagatctcattacagatggttgtgaaccaccatgtggttgctgggatttgaactcaggacctctggaagagcagtcagtgctcttaaccgctgagccatctctccagcccccacacctagtgtttttgcacgggttctgaggatccaggtcaggccctcaagcttgcaatagaaacacttcgccaactgatctgtagccctgtcccttgctgctctctgtgtataagaatgccagccaatttgcatcggagcccactggggagggctcgttttaccttgtctttaaaggtgctattgccaaatacagtcacattctgatgaatgtggtggagcaggcctggaattccagctatggcagaggctgaagcaggagggttacaagctcatataatatagtgatttcaaggttaagcagggcgatttactggactttgtcttacaataaaaagtcaaaagagaactagagatgtaactcgttgatagaatgctttccaagcatgcacgagagcctgggtcaatccctagcccacttagaaactacaagtggtggggtgcacctgtaatgcgggaggtggaggcaggaggattagaactccaagccatcctactctatacatggtgagttcaagaccagctggggctgcatgaaccttgtctggagtgggctgtaatggtacagcacctacctagcaactcaagaagcctcggactccatcgtcagacaggcacaccgcagggccctggaggtgggaatgttagcaaggagatctgggagcagttcagtggatgagtgtgctttccacccggaacatcctgggttcccacggatctctgactgctctccttctcctgactggcccccgctgtaggtgccgtctcctgggctatctacagcagccgcgcttttggctttgagtgagttcttaagtacatagcaaacaaaagcagaaacagaccgcaagggatggctagaagcgccagacacatggcaaacatcagatcaggctctgaagacacccagcagagaccgactgtacaaacacacgtgcacaccctggaatgggatgaagaaaagtcagcctgactcccaaagccagtagctcagagagtgtgaacagcacatggctatttagccacaggatgtacattatagccactgggaataccagggtagctaaaggtttggcttcagggctctaaataatctcttgtcactctcgaaaaatgtttttccaggatacacagaagcaaatgcacagatcggagatgactcccacgggggcaggcctgaaggccaccatcttctgcatcttgacctgggtcagcctgacggctggggaccgcgtatacatccaccccttccatctcctttaccacaacaagagcacctgcgcccagctggagaaccccagtgtggagacactcccagagtcaacgttcgagcctgtgcccattcaggccaagacctcccctgtgaatgagaagaccctgcatgatcagctcgtgctggccgccgagaagctagaggatgaggaccggaagcgggctgcccaggtcgcaatgatcgccaacttcgtgggcttccgcatgtacaagatgctgaatgaggcaggaagtggggccagtggggccatcctctcaccaccagctctctttggcaccctggtctctttctaccttggatccttagatcccacggccagccagctgcagacgctgctggatgtccctgtgaaggagggagactgcacctcccgactagatggacacaaggtcctcgctgccctgcgggccattcagggcttgctggtcacccagggtgggagcagcagccagacacccctgctacagtccattgtggtggggctcttcactgctccaggctttcgtctaaagcactcatttgttcagagcctggctctctttacccctgccctcttcccacgctctctggatttatccactgacccagttcttgccactgagaaaatcaacaggttcataaaggctgtgacagggtggaagatgaacttgccactggagggggtcagtacagacagcaccctacttttcaacacctacgttcacttccaaggtgaggcaaacacttgggtcactggtcctggttgtaccagaggcagcactgggtttccatgacatccccatgggtcatgtgtgagatggacagaggtgactggtttctgcctctgtgtgcctggtgaagccctggaggctaaaaaaaaagcaaacttggctcaagatggcacagcctctgcctccctacccaactgcctttctgggctgtgtcatactcctgagactacggtggttgacccccacaggaacacccagctcacccgctcacagggggagctgcacacatatccctgtccaggaagatgctcatccattcgatgatctcttactacttgtaagaccgggcaggggtctttgaggggcttgagatcctgatcctccaggtttcttattaaacatcagcaagtatggagacccccaagggcaaatgcctgaggcaacactgcagggaacattgctacctgtgaggtggaaaagggagtgagctggaacaggctggaggaggaggccaagggcaaacatccttgcaaagccttggtaggtcagggtgggagtgagggtggaggggctcagaggtggcacaccctgtgaggatgctctttctccagtaggctgagcagggcagttttatttctagaaggtcagactttggcccctgacagcactgcaggggtgtgtgtgtgaggggtccatataccacccaaaggtgtgcagtggtccagggacactgttctgggaacagatgctagggtcccagatcttcaaattgaggttactagaggaggggtgcattggtggggccagcaggaagacatcccggcatggggtagagggatcagggatgactggccactgtgtcaatatctttgcagtttataaattcacatggggaaagaccttggcctgccttgagactgggaaagggggaggccagaatgtcgagggaacgagacagctctgagactggtgaagggtggctacacctgtgtaagttggcgaggagcctggaactgagccacagtcctcatgagaaaccctgggaagacgatcactgagaaaggagcacttctaaagggcagcagtgccggtcatggcttgctgtagaggactgcagaaccagccgggagggaatagagagaggtgaagggaagggatggaggaggaatgggaggagggaggtaaggagggaagaggagaagggtgggagaaggaatgggaaggaggaggaaaaagggggaggaatggtaggatggaggaggaggaaggggaggaggaagagaaggagaaaagggggacgaatgggaaggaggaaaaaaagaggaggaatgggaggaagaaaggaggaggaatgggaggaggaatgggaaggaggaggaaaagggggagtgggaggagggaggaggaggaaggggtggtggaagatgaggaggaatggaggagaaatggtaaggaggagaaaaaggagaggaaggggaggaatgggaggagggaggaggaggagggagaggaggaagaatggggaggaataggaaggagaaaaaaagaggaggaatgggaggaggaaagggaggaggaaggggaggaaggggagaaggaatgagaggagaaaagaggaggaagaatgggggaagggatgggaaggagaaaaaagaggaggaatgggaggaggaaagggaggaggaaggggaggagaaaagaggaggaaggggaggaggaggaaaaaggtcaaggaaaagaggaaggggaaatggaggaagagagagagagagagagagggaagaaaaggaaggagatgaggagggggaagaggggcaggttccacgacactaggggacatgactgggagaagtttacgcaaaggagaaggagaaaaatgggttgtgccagctctcagagacctggatgctcacccgtgtcctgtcctcactgctgcctggccttttctgcctatctccatctgccagaggcctgccctcttcctcacacccatgaggttgcagatgtgcctcagaaactccaggtccccttagcaacagccttggggacagtttttgtatttgtatctagtatttgtatctagtgaggcctcgaggccttgggcttgtggtctctccagtgttctttgtccagtacttgatgtccccggagcttgaatggccctggcaggttccatgacactaggggacatgactgggagaagtttacgcagcccacagtagggctggtgagcagagaggagtctggggagcacggggagaacggggaacagacagcttcgggtcgctctcgaagccgtgccccatttctcatttctcttttatgctagagacaaaaattcagtgctttccccctaaactgaagattcctgaggcccctgaagtgattgtcagcgtctctctcatgtgagcttcgtgtgtgtgacagggtctcactgagtaccttggactggcttggactctgaaatacttcttacctcagtgttctgagtgctgggatcacaggtgtgagccactctagtgacctttagggaagggctttttttttttttttttttttttttttttgagacagggtttttctctgtgtagccctggctgtcctggaactcactttgtagaccaggctggccttgaactcagaaatccacctgcctctgcctcccaagtgctgggattaaagcaggtgccccaccactgcctggcttagggaagggctcttatcactcaccagagagggctctgaggtcaagaccattctacttccacgcagtcggaacccagcaaccccttcttgctggagttacactggggacccctgacttcatacagcaggaccaatcagagcaggtgctacgtgagccaagcaagcctgcccctcaatctcagccctatctccctgaactcctaaatgtccctttcgtggtaaatgggagggcttgggggcatgtgtgagccggaattcagtgctgggtagggcagtcataaggagaatgcaacaatgtctgagtgcagtgctggctgagccggtggcatcaggctggggtggctcagcttggaacgtgtttccttcctgtcctgtccctagttccctcctgccctcagcagctgggatggtggtggtggggattcatccctgccccaaggccctgtgttcagatgcatcggtcactgtagagttacacctgtctccagggcaagggatgctggacagtggagtcttgcttgggccaggagcctgggcagctgcattcaaatatgctgtgtgtgaggcaggggccaaagtctctagcaacctgttgtgagtaggcagagagaaatggctctggtgatgccaccgagttgggacaggtcatggcaggtttgtctaatcctgtcgagtgtccaatcagagcaggccccatgtgaaccaagggctctgggcacagttggggtgtctgtgtcggcccactgatggcctcaaaggactccatggtgacacagagagagcaggactccggaagccgaaccaggggctcagtgttgctgggaatcgaatgctgctctaaaaatatcttttttttttttttttttttggtatatgtgtgtggtgtacgcgtatgcatactgtacagaggaacaggttgagtgtcttctattttttccttatttatttatttatttatttacttatttatttatttatagacagcatttcttaccaagcctggggcttaccatttcttctaggctggccagtgagctccaagggtccttctatcccctcatccccagtgctggcattatggacctgcataaccatgtctcactggcacaaggggctggggctctaggctcaggtcatcaggtgcatagagcaagtagttatccatggagccatctctccagaccataatgtctaaggtttgaaaaagtgagtttgcaaaccagaacgggtctcggttgagcttgcttggtcagagaggaacagaccgagttctccaggtgtcagatgtgagacctcggactccacaccatcatgggctgtgtctcctgctgctcttggggatcgctgtaggggaaattacccaagtggctgcaatttacaaaagtatcagaacacctgagggtgaagtctccagggcagtgcaggcgagggaggaagctgaggcaggagccagggtggaggagtcccgcccctgcttccccacccctgttgagcttctctttggtctctgctgctttcaggaacgatgagaggtttctctcagctgcctggagtccatgaattctgggtggacaacagcatctcggtgtctgtgcccatgatctccggcactggcaacttccagcactggagtgacacccagaacaacttctccgtgacgtgcgtgcccctaggtgagagagccaccctgctgctcatccagccccactgcacctcagatctcgacagggtggaggccctcatcttccggaacgacctcctgacttggatagagaacccgcctcctcggtaggaagtgtgcggggaaccttggggagtagagctgcatgaatggcatctgctgcatgctcacacatcagtctgtccaggtctgtccatgtaaacagcatgtcctgttaccaccccctccctccctcctgccatcatgagcagtgtggttgtgggtatggctgttatctgtgaggctatggatattcccttcctccacagggaggcagtgagccaactggggctggtttagggtttctattgctgcaacgaagcaccatgaccaaaaagcaagttggggaggaacgggtttatttggtttacacttccagatcatagtccatcactgaaggaagacaggacaggaactcaaacaggacaggaacctgggggcaggagctgatgcagaggccatggagggatgcagcttactggcttgcttcccctggcttgcccagcccgctttattatagaaccaggaccgccaccccagggatggcaccacccacaatgggcagggcccttctccatcaatcactaattatgaaaatgccctacaggcttgcctaatctggaggcattctcttagctgagactcttagtttgtgtcaagttgacataaaactatccagcacagataagggccattatcttattagcgttttggttttagagagggtctagcactcgtggtaatccccctgcctccacttctcaagtgccagggtgatagggatgaggtgctctgcctggctaatggcctgttagtcaaaggcatctttggcttttccagggaaggcactccttatgcagccaggctgagagcacagggcacacagctgcccaccaccagcaagctctccacagtcgatgttactggtcctcctcctgtttcaggaggtttttgttgtgtttgtttctgagaagttccttggggtgacaatgtcctgtattgttgtgtgacccttcttggggagatgcaagcaaatatatattcactccagatagggcaccaacttcagacccaagaatcaatcccttccgagtagaattcggtcattcagagcattcattgcagttacttcgttgggcagcatgggcgaccactcacggaagctgcatccctggagctccctgcacaacctggaggcagctctaccagtcccctttccccagcagtcatttcctgattataccatgctgggaggatcctgtgcagcttccagagcagctttctccagctgtgtcttatgcacccccttcccgcagggactgtttcaactcaggacactgactggctgagcgcttagtctctttttcagtgggtgggagcttcccagccccctaaaggctttatctccacagggccatccgcctgactctgccccagctggaaatccgaggatcctacaatctgcaggacctgctggctgaggacaagctgcccacccttttgggtgcggaggcaaatctgaacaacattggtgacaccaacccccgagtgggagaggtgagtgctgctctggctgtgtagcagggaagggggggtggatggtcacacggaggaagctgtgcccaagaccagcagggcaggaaggaaagagcagcaggcactgccaggactctggctcccagtgacatggggaacagcggtctcccttggctctcttggcatctctagttccaggcccatctctgttcctgcttagtccagccttgcatcctccataaagctgtggtagatggggaatgccccatgccctggtctgcagtttctttggggagcatctcaaccatagacagagagctatgtaaaacagaggatgtgcctggattctgttggcattgccaaggatgctgagaaacaggggagtcactgtagaagacatgaccccactgtgctgtttagccaaatatgggagagtaccaagatggcaggtgaacaaggggacaggggcatgttgagcagcagcctctatgggagctgggtgttggtgcccagcccccacttagaggacaggtgtgtccacacttccttctggcaggctggacagtgtccccctttggggcggttgagccagtccctggcctgtaacccctccttaaggagagatctctctgctttgcaggttctcaatagcatcctcctcgaactcaaagcaggagaggaggaacagccgaccacgtctgtccagcagcctggctcaccggaggcactggatgtgaccctgagcagccccttcctgttcgccatctacgagcaggactcaggcacgctgcactttctgggcagagtgaataacccccagagtgtggtgtgaggccttgtgcctagccatggagacaaggccggtgtcggagaaccgttctgggcaaaactcagtgctgtcacccctggctccccatcacgccttgtagcgcggcagaggccgtctccttggagactgcgctgaccgagaataaatgatgagcagcagagcctcctgggatgtgggtttgtttggatactggggtgacagccagaagctggcactctgcacaggactgccactctggaagaaatttggaccaaaaaactgtttgtgacaccaaaaagcacccccccttttttttatttgaggacagaaattgggttttaacattaaaatgcacattatccccttattttgggtctgtaattagctcactgagtattagaccatgacgtccagagtcctacaatgtcttacgacttcttatccctggcaccatgatttgaaaacatattcctcactgtgctgagaattgtctttcagtgataagctaaacacagtattatgggcagagcagacatgcttagtttagctgacaggctgagcaacattcctgaagctcttgacaaggaggccaatgtgtgccggccaggtggtagaggagagggctgtcaccaagaggccttgagaaaggacctgggcggaggggccttcctttactgctaccacctctctgcctgtccccttgagatcaaatctgcagcttccagatctaggagtgtgctgaggaaggagcaagacaggccgaggtagtggtgtgtgcgtgtgcgtgtgtgtgtgcgtgcgtgtgtgtgtgcgtgtgtgtgcatgtgtgtgtgtgcatgtgcgtgtgagcatgtgcgtgtgagcatgtgcatgtgtgcgcgcgcatgtgtgcacgtgtgtgtgtgtgtgtgcgagcgcgtgtgcatgtgtgtgcatgtgtgtgtgtgtgtgtgtgcatgtgcatgtgcgtggcaggctgcctcctccctgtacagactgggaagcaggtgagagcattgtagcatgctagcctcaggcgaaggccagagagtacagtaaagcccacagggcacagacagtggggaatcctgtgttagaacatggagtcacctctctggcctaagctggagtttgactctgtgtgagcatagcctaacctcacagacggcttctctggagcttggagctgagaccatagggtcctgtaagtacaggtggggcataaagtgtgttcctgtgaccggggtccactctgaggcccggcttctctcatcctagtcctgttgcctcctgaaaactcctctctggcttcctgtacctcaaaggcacttaccctgtgcgtgtggcaatctagctagtccaacccctgcttgtgccaggcaacatagtgagggacctgtgtaccatggttaactagaggccaactcctcactctccctggcctatgggcttacagggatgctaagaaaggccctggtccagagggaggtggcgtgaacttctcatggctgtgacaaaatagcagacaaagcaatgtaagggtttattctgggtcatgcctgagtggtggggagagtgtaggaggggggcacaagacagctggccacttacagtgaagacacacccatggagatcacacatacacacacacacacacacacatacacacgctataccacagccacacatggggaaggggaggggccgaggctggtgttcaactcattttcttagcccatgggatgctgctgtcaattcttacgaggtatctcccacatcatttaaccccacctagaaactgcctcacaggcatatccaggggcttgtttcttggatgaccctaaattctatcaggctgataagatgaaccatcctaggagtccatgggtgtcttagtcagggtttctattcctgcacaaatatcatgaccaagaagcaagttggggaggaaagggtttattcagcttatacttccacactgctgttcatcaccaaaggaagtcaggactggaactcaagcaggtcaggaagcaggagctgatgcagaggccatggaatgatgttgctcactggcttgcttcccctggctcgctcagcctgctctcctatagaacccaagactgccagctcagagatggtcccacccacacgggacctttcccccttgatcactaattgagaaaatgccttacagttgtatctcatggaggcatttcctcaactgaagctcctttctctgtgataactccagctgtgtcaagttgacacaaaactagccagtacaattgaccccttgtcaacttgacacacaaacacatcactagtaagcctcaacccttactttcttattcatccccaagatctaaataactttaaaagtcccacagtctttacatattcttaaaata

SEQ ID NO: 13 - DNA, 1958 bp, Rattus norvegicus
  CDS (201)..(1634)
ccttgctccatcttggctaagcctggattcccatggtcccccgacctgggtcctcccccagcctctgtacagagtagcctgggaatagatccatcttcaccccctcgagtataaataaggctgcttggttcaccaggggatagctgtgcttgtctgggctggagctaaaggacacacagaagcaagtccacagatccgtgatgactcccacgggggcaggcctgaaggccaccatcttctgcatcctgacctgggtcagcctgacagctggggaccgcgtatacatccacccctttcatctcctctactacagcaagagcacctgcgcccagctggagaaccccagtgtggagacgctcccagagccaacctttgagcctgtgcccattcaggccaagacctcccccgtggatgagaagaccctgcgagataagctcgtgctggccactgagaagctagaggctgaggatcggcagcgagctgcccaggtcgcgatgattgccaacttcatgggtttccgcatgtacaagatgctgagtgaggcaagaggtgtagccagtggggccgtcctctctccaccggccctctttggcaccctggtctctttctaccttggatcgttggatcccacggccagccagttgcaggtgctgctgggcgtccctgtgaaggagggagactgcacctcccggctggacggacataaggtcctcactgccctgcaggctgttcagggcttgctggtcacccagggtggaagcagcagccagacacccctgctacagtccaccgtggtgggcctcttcactgccccaggcttgcgcctaaaacagccatttgttgagagcttgggtcccttcacccccgccatcttccctcgctctctggacttatccactgacccagttcttgctgcccagaaaatcaacaggtttgtgcaggctgtgacagggtggaagatgaacttgccactagagggggtcagcacggacagcaccctatttttcaacacctacgttcacttccaagggaagatgagaggcttctcccagctgactgggctccatgagttctgggtggacaacagcacctcagtgtctgtgcccatgctctcgggcactggcaacttccagcactggagtgacgcccagaacaacttctccgtgacacgcgtgcccctgggtgagagtgtcaccctgctgctgatccagccccagtgcgcctcagatctcgacagggtggaggtcctcgtcttccagcacgacttcctgacttggataaagaacccgcctcctcgggccatccgtctgaccctgccgcagctggaaattcggggatcctacaacctgcaggacctgctggctcaggccaagctgtctacccttttgggtgctgaggcaaatctgggcaagatgggtgacaccaacccccgagtgggagaggttctcaacagcatcctccttgaactccaagcaggcgaggaggagcagcccacagagtctgcccagcagcctggctcacccgaggtgctggacgtgaccctgagcagtccgttcctgttcgccatctacgagcgggactcaggtgcgctgcactttctgggcagagtggataacccccaaaatgtggtgtgatgcctcctgtgtagccatggagacaaggccagcgtcagagagctatcctgggcaaaaatcagtgccttcacccctggcttcccgtcactccttccagcaaggcagaggccgtctccttggagatggcgctaactgagaataaatgatgagcagcagcctcctggggtgtgggtttgtttggacactggggtgagagccaggagctggcactctgtataggaggactgccatcctggaaaaaaaaaatggaccaaacaactgtttgtgaaataaaaaaaaaaaaattccctttttatttgaaaaaaaaaaaaaaaaaaaaaaaaa

SEQ ID NO: 14 - DNA, 783 bp, Rattus norvegicus
tggaagggacccagcggaacatcgccctggccttgctccatcttggctaagcctggattcccatggtcccccgacctgggtcctcccccagcctctgtacagagtagcctgggaatagatccatcttcaccccctcgagtataaataaggctgcttggttcaccaggggatagctgtgcttgtctgggctggagctaaaggacacacagaagcaagtccacagatccgtgatgactcccacgggggcaggcctgaaggccaccatcttctgcatcctgacctgggtcagcctgacagctggggaccgcgtatacctccacccctttcatctcctctactacagttagagtacctgagccctgctggagaatctcagtgtggagactctcccactagtcaacctctgatactgtgttagttcaagacactggtctcttaagcgcaacttggtgtcctgcttaattggcttagcctgcgcccttttgcattttcatttagatgttgcttttttactgatttcttcacacccacgcctatgtcatcttccttcttctctacatacttgatttcataaataaataaatttatatctcctcttgtttacctaattttttttctatccatgcacttgaattacttctctcattttttcattttgttcctcttctcatcctattaagattcaattccacctcacttattagcactttcctaccacttttcttttcctccatttctagttaatacatcccacctgcacatacttaataatttctacatttc

SEQ ID NO: 15 - DNA, 553 bp, Rattus norvegicus
  misc_feature (227)..(540): n = A,T,C or G
cacgaggggaagcagcagccagacacccctgctacagtccaccgtggtgggcctcttcactgccccaggcttgcgcctaaaacagccatttgttgagagcttgggtcccttcacccccgccatcttccctcgctctctggacttatccactgacccagttcttgctgcccagaaaatcaacaggtttgtgcaggctgtgacagggtggaagatgaacttgccactanagggggtcagcacggacagcaccctatttttcaacacctacgttcacttccaagggaagatgagaggcttctcccagctgactgggctccatgagttctgggtggacaacagcacctcagtgtctgtgcccatgctctcgggcactggcaacttccagcactggagtgacgcccagaacaacttctccgtgacacgcgtgcccctgggtgagagtgtcaccctgctgctgatccagccccagtgcgcctcagatctcaacagcatcctccttgaactccaagcaggcgaggaggagcagcccacagagtctgnccagcagcctggc

SEQ ID NO: 16 - DNA, 15751 bp, Rattus norvegicus
gaagccagccatggaggtcccaggatgcagatggaggaagctctgcctccctctcacactctttcctagagagtgaaggagcaagggaccatgggggcttcaggccagtcagctaaccccctcagagcagacggaggctacacagccagcctggaccaagagccctgttgtctgacttgccactccaggtggagtaaataagtctgtggagtgatgaccagccagcagagactgacgcagaggaggctcgtgctcagacagccttagtagcaagggctacctgctctcagtacagtgcggttacacctcagtctggtcccaggcttcctcacgctggacaattggctagaacagctccagcagtcacgttgcctctgctgactggtttctcatgaggcccacagaaatagccagggtcagaatggctgacagtacaggacctcgtcagatggcaagtacatcttcattcctggctgtgttcctcagcttgggaattctccaaaccccagggcaaaggctctttactgaggctcccctgcatacagggctggcgacatccctgccatcactggccagctccatagccgcccttctctcttcgcagagggctgaggctgaaagttcctttggatcgcatggccagtctctctggtcactacccatctcctgagatgaccgtgaggccttcatctcccagacttctcacaggggacaacaagatgttcagtgagcttaagaatccagccatctagtgcttgcctagcaagggcaaggccctgggttcggtccccagctccgaaaaaaagaaaagaaaaaaaaaaaaaagaatccagccatctgtggtcagaaacagagaggagagacctatctatcactatacttgggaactctactcttgtgtccccagcacagggcctcctgctgaaccccaaagctactaaggcatccacacactcctctaggaaatctatgcaccactgccctttcctgagctcttactccttgtacatcccttggtcctctggcatccttagtcctgagtgcagaccgtcatggctgcagcctggagtgcagaccgtcatggctgtagccttgatgcctccaacaactggcttcccactgagatagatcttctggtaattattttacatctgtgcgcagagaaggtcactctctagagcccagctcagacaccatcaaatcctgcatggtccatctagtttcttctgagccagggactgctctgccaatcctccctttccgccttcactttctagtgccactttagggtaaaggcaggttgtgcctgtgtcaactccatggccaagagattacttgacgtactggatgcaaggcaagttagtcgcccacccatggtggactacaggggcaaccgtctaccagacctactcctgctgtcttctcagctcccacccttccttgggatccacccgtctcattctctaaacctccatgactattccacactcaagggcggtgctctgaaacctccagcctccaaaacagccgccttccatccacaagcccagaacattttgtttcaatatggctttaccacagttgggatttcccaacctgaccagatgtgctagaagtttcccaaaggtgcgggaagggactggaagcccctgtgcagaacagacaactaacgtaattacaaccattcaaggtcgtccatcctgagagccgctgatgacttatgagaggttcatctcctgcaggtaggtcccttcctggcatgtcctgagaggcttataagcagatcacgaggggcttacgtcccaaggtctgcatggtggcttcagttgtcacaacccagccagtcctgtgactgtgatcccagctccggggaactggggtaaatatgtaactcaacgccagcccttttttttttttttttttttttttttttgcctgaagctcatctgccactagggcttcctggaagggacccagcggaacatcgccctggccttgctccatcttggctaagcctggattcccatggtcccccgacctgggtcctcccccagcctctgtacagagtagcctgggaatagatccatcttcaccccctcgagtataaataaggctgcttggttcaccaggggatagctgtgcttgtctgggctggagctaaaggtaagcagagccctcttgggtcctttctggtccttcgtgggatctgactgggcaggagctgagcttcagggaagaagaggaggcaggggttgggtagagacactggaaggggttggatggcatttgtcctggagtgaggagctagaagtctgtggtggcaggcgcttcagatgtgaccactgacttgtgtctcctctcccgaaaggctagatgtccaggtaagctgtcccctgcgggtcaccactctgggctcaccggcaactgcttgtgttttgttaggggatggccaggccttctgcttcccgtgagcccagatgggtcctcaaaggcagagggactttcacaggctcagcactagagtggctgagtccgctgaacctcttccagctcgaagggagaacaaggctccattgtttggactgcaagcagcaaggagggacaggtcccatggtcatctcaagtagcatctctgtccaggctcagtgctggggcctgggtcttgtggcttctcaccacgtcctgtagcgtctcaacttacccctctgcaaacaggctccttgtggttcgaaggttctctagggctggggataagttaggagcacccagtagcaaggaagaactttgataatgtcaagctaagcaaaaaaggtagctccttattttcagtgtgtgtgtgtatgtgtatgtgtgtgcgtgtgtgtgtgtgtacatgtgtgtgtgcatgtgtgtgtgcatgtatgtatgcacgtgtgtgtgcatgtgtgtgtgcatgtgtgtgcatgtgtgtgcgtacacgtgtgtgctgtgtagtgtatgtgcatcttatttattcatttatttttgtttttcgagacagggtttctctgttgtagcctttgctgtcctagaactcactccgtagagcatgctggcctcagattcggagacctgtctgcctcggcctcctgagtgctgggactaaatgatttttttttttaaattttaagacagggtcttactgtgtagctctggttgccctagaactcacaaaattctgtctgcctctgcctctgattctatcaccatgccctgcccaaggtatgtgtgtatttatatgaatgtgacatatgtgtatgatgtgtgtgtgtgtgtgtgtgtgaagtgtgtgtggtatatggggagtgtggagactcaagagccatgaacacagatagaggccagaggaggacatcagctgtcctgccaggtactgtcctccttagtctctggagacagggtcccttactgaagttggggctaggctggcagacaggaagcccgggagatcatcctgtctcctccgtccacgtctctggagttgggggattatgtggtcatgactggtttgctgagcctgccccgggctcctcagttccttctttctttcaagaggaagtgtgcgtcgtgtctagataccactcatggagtcaggtgtcaggtctgagtcccagctgtgtgacctggtgctggccacaagacttgtctgttcctctacaaaacagcactgacgactccacccctggctgtcccaggatgaccttagggaggtgggctaacctgtggctgctcggtagagagaaggcgagggaggggtgtgggagcccagggtagactttgagaggtgaagaggatgcacttagggtgaacttgactggcagcagcttatggctgagagcaaacagtcacttgtgcctgaggacagtggtgacgcgcttcctgggacagtggctgtggcacagcttccccctccccccctggacagaggccattcacggggacagcaatatgggaagcattgttttcacctcgatggctcaccctgcccatgggggaccgaggcaggctcgaccctactgtgccttggtttataatagcacagtgaacaggatgccgtcattcctgggaggaatgggagggccagtgggtacaggcagagttttctactctgcttttcagaaccacagaactttgggaacattgagtggatcagatgcatgtgccaggagggtaaagtgcaggacagtcagaagccaaaggtgaccagtgggatgaccagagcagagcaaagagtaccttgcctgtctggaggcaatattctatgtgtggcccaggccaagtgccacgacccgggtgtggggggccagggtgacctcggagctttgcaacaccagagtgacatcctgagatcattaacttaccaagagaaaagttttgtcttggctcgtgggactggagtcacaggtcaaattcagacagacttgttgctcgggaccttctgggggtgttggctatcaacagcaacagtggcatttggccaaacaaattgcttcccatgtagcaaggaagcaaaaccagtgaaggaaagacgcaggtcctgagatcactcttttttttttttttttttttttttgggtctttttttcggagctggggaccgaacccagggccttgcgctttctaggcaagcgctctaccactgagatgaatccccaatacccctgcgatcactcttaaaggcctggccctactggactcagggcccccactaggacttgcatcctaaatgtttacagcacctcccacggaccaatgctgggcattaagattgtcacacaggggggttggggatttagctcagtggtagggcgcttgcctagcaaacgcaaggccctgggttcagtccccagctccagaaaaaagaaaaaaaaagattgtcacataggaaccttgcgagggtttgccgagcacagcggaaggtgggagaagcagaaaggaagggtcaaggtgacctccagtcagggttgaggcaccaaggcccagcggtggtcgccatagtgaggtaaggggtaggaagccacacatcaggctggttccttctattgaagcaagagcacgtggttcacacggtcagaactgaagggcagatagagctcgcctccccacaccaggatcagaggagtaagacgtgggtttttcagagtcagctcaggacccttctgctaagatcgcaggctaactctatgaactgtgtgtggtagctaaggctggagctgaggttcaaggagaaaggctatccagttcaatggaccacctcatccttggtccatgactggccagagccccaagcccttcctgtgctccacagccatctgggcagttggcaaatcaacccccggctgaagatagactgttgaagtgagcaggctgggcagacaggccagtcatggagtcttctgtagctgctgtgtgggatccagtctgcctggagcacgtaacgggcaggaagggtctggccttgggcaaggcctttggttcccaattctggctgcaggctaggctctaatggacactaaagtcaatcaatcctagggcacacccatgtgggtacaacggctccacagagtgggcagtcctcatgtccttctctagggatgagcttatagcctttgtcacttgtatgcctggttccaccaggactatgctacggtctagtatgataacaaaaggaccttcagaaacccaaagcaaggcatggtcagagccaagttcttctgaggcctctctctgcctttggcttgcagattccctcctctccctgtgactctaatagatttctctgttcctgtatcctgatatccctttgtgtgtgtgtgtgtgtgtgtgtgtgtgtgtgtgtgtgtgtgtgtgcatatgtgtgtttgtgtgtgtgcatatgtgtgtatttgagtgtgtgtgtctgagtgtgtgcatatgtgtgtatgtgagtgtgtctgtgtgtgtgtatatgtgtgtgtgtgcatatgtgtgtagagcctggatgaccaccttttgtgtcatgtcaaaattgtgtccatctcctttgagacagggtctctattggcttagagctcacacattaggctagattaatttgccagagaaccctggggagtcaaaccccaggaatctacctgtcttcggtgcctgagcacggaggttacgtgcatctgctgccatacctaaaggtttattatttattttatatatatgagtacactgtcgctgtcttcagacacaccagaagaagacatcagatcccattacagatggttgtgagctaccatgtggttgctgggatttgaactcaggacctctgaaagcactgtcagtgctcttaactactgagccatctctccagccccacacctactagttttacatgggtcctggggatccagcccaggtcctcaggcttgcgatagaagtacttcagcacctgatctgtatccctgtccctccttgccctctgtgtagaaaaatgccagtcaatttacatccgagcccaccaggagggcctcatcttacttggtcgtgtctttaaaggctctgtctccaaatacagtcacattctgatgaccgtggtggagcaggcctggaatcctggctatggcgggggatgaagcagaagggtcacaggctcaaagctgcctagaagagagagtggtttcaaagttagccagggcgacttattggaccttgtcttaaaaaaaaaaatttttagtcaaaagagtgctagaggtgtggctcgttgggagaatgctttctgatcatgcgtgagagcccaggttgatctccagcccacttataaactacaaggggtggggtgcaggagctggaggcaggaggatcagaacttccaagtcatcctaatctatctaggggttcaagaccaactgggtctgcatgaaatcttgtctggggtgggatgtaacggtaaagcacctacttagcaactcaagaggccctgggttccatccccaaacagccacactccagggtcccggaggtgggagcaactcggcgggtgacagtgcgttccacacggatcatcctgggttcccatggatctctgactgcactcctcctcctcctaactgacccgagctgtaggtgcctcctcctgggtcgtctacagtagccttgcttcgagccttgagtgagttcttaagtacatagcaaacaaaagcagatagcaagggacgggtagaagcaccagaaatacagcaaacatcggatcaggctctcaagacgtccagcagagacgactgaacaagtgcacccacacaccctggcatggggatggggagtcagccctatttccaaaagccagtagctcagagagcgggagcagcacatggcagtttagccacagagtgcacattacacacagtcatggggaataccagggcaactaaaggtttggcttcagggctccaaataaccacttttcactctcacaaaaaacccattattccaggacacacagaagcaagtccacagatccgtgatgactcccacgggggcaggcctgaaggccaccatcttctgcatcctgacctgggtcagcctgacagctggggaccgcgtatacatccacccctttcatctcctctactacagcaagagcacctgcgcccagctggagaaccccagtgtggagacgctcccagagccaacctttgagcctgtgcccattcaggccaagacctcccccgtggatgagaagaccctgcgagataagctcgtgctggccactgagaagctagaggctgaggatcggcagcgagctgcccaggtcgcgatgattgccaacttcatgggtttccgcatgtacaagatgctgagtgaggcaagaggtgtagccagtggggccgtcctctctccaccggccctctttggcaccctggtctctttctaccttggatcgttggatcccacggccagccagttgcaggtgctgctgggcgtccctgtgaaggagggagactgcacctcccggctggacggacataaggtcctcactgccctgcaggctgttcagggcttgctggtcacccagggtggaagcagcagccagacacccctgctacagtccaccgtggtgggcctcttcactgccccaggcttgcgcctaaaacagccatttgttgagagcttgggtcccttcacccccgccatcttccctcgctctctggacttatccactgacccagttcttgctgcccagaaaatcaacaggtttgtgcaggctgtgacagggtggaagatgaacttgccactagagggggtcagcacggacagcaccctatttttcaacacctacgttcacttccaaggtgaggcaaacgcttgggtcattggtcctggctgggtttctatggcatccccatggttcatgcgtgagagggacagagatgacaggtttctgcctctgtgttaccagtgactccctggaggccaaaaaagataacctgactcaagatggcatggccctgcccaactgcccttctgggctgggtgatgctcttgaggctagagtggctgacccacgcaggaatacccagcttactccctccctcacacggggagctgccgtatatccctgtctgagaagttgcccatctgttctatggcctcttactacttgtaaaccaggcagagggctctgaggggcaggagatcctgaccctcctggtttctcaagagacgtcagcaagtatggagacaccccccccccccgagcaaatgcctggggcagtactgcagggaacagcactaactgaagaggaagggaatgagttgtaacaggctggagaaggccacaggcagacatccttgcaaagccttgggagaccaggttaggagtgtgcaggggctgggctcagagagggcagaccttgggaggatgctctttttccagtgggctgagggggacagttttattcctaggacattggaccttggcccacaacagcatggcaggtgtgtgtgtgtgtgtgtgtgtgtgtgtgtgtgtgtgtgtgtgtgtatgtgcagggtgtgcagtggtccagggacaccatcctgagaacatgtgccagggccccaggtcttcagattgatggttactaggggaggggtgaccaatggggccagcagcagggagacatcacagtctggggtagatgtagaaggatcagggatgactgggccactctgacaatatctttgcagcctataaattcatgtggggaaagaccttggcctgctttgagactgggaaaggtgtggggggtagaatgtggacggaaagaggtaaccctgaggctggtggagggtggctacacccatgtgagtcggaaaggaacccggaactgagccacagtccctgtgggaaaccctgggaggactatcactgagaaaggagcacttctacagggcatcagtgtcagtgatggcttgctgtagagaatgcctgttttcacaccccagactctggagccagcagggaacagaggagggcgaagggaaggagtggaggagggggggggagagggaggtgaggagggagggggagaggagtgagaacaggaacgggaaggaggaggaatgggaggaagaaaagcagaggaaggggaaggagaaggaaaagaggaggagtgggaagaggggaaggaggattgggggagggagggagaggaggaatgggaggaggaaaagaggagaatggcaggaggaagggggaataaggggaggaggaagaggagaaagaggaaagggagggggaagtggaggaagaggaaaaagagggtgggaaaatgaaggaagaggaagagagggagagggaagaaaagaaaaaaggagacgaggaaggaaaagaggagaggttgggggtggggcaaaaaatgagttgtgccagcactcagaggcctggatgctcacttgtactctgtcctgatctcactgctgtttggcctttcctgcccatctccatccgccagaggcttgtcctcttcctcacacccatgaggctgcagactgcctcacaaactccaggtccccttagcaatagcattggggacagttaagcatttgtatctagcgaagcctcaaggccttgggctcgtggtctctacagtgttctttgcccagtacttgatgtctccagagcttgaatagccctggtgggttccatgacactaagggacatgaccaggggaagtttactcagtccataataggactgctgaacagggaaggatccggggagcatagggagaacaggaaacagacagcttcacatctctctcaaagccttgtcctgtttcacatttctcatttcttttttcttcatttatttattatgtatagagttctgcctgcaggtacacttgcacaccagaagggggcatcagttcccattacagatggttgtgaaccaccatgtggttgctgggatttgaactcaggacctctggaagagcagtcagtgctcctaaccgctgagtcatttctccagcccaatgcctggctcttacgagggactggggctgtaggtgcaggtcatcaggcctatagagccaagttaccatggagccatctctccagaccataataatgtctgaggttctaaagagtgagtttgcaaaccagaacaggtctgggttgagcttgtctgttcaggtaggagcagactgagtccctcgggtgtcccatgtgagacctcagactccacaccaacatgggctgtgttttttgctgctcttggggtctctgcagggaactcaagtggctacaattagaagtagcatcagagagcacctgaaggtgacatctccaggtcagtacagcatcacagagggagtagaggtgaggaagctgaggcaggagccagggtggaagagtcccagccctgcctccccatcctacccccgttgagtttcctttggtctctgttgctttcagggaagatgagaggcttctcccagctgactgggctccatgagttctgggtggacaacagcacctcagtgtctgtgcccatgctctcgggcactggcaacttccagcactggagtgacgcccagaacaacttctccgtgacacgcgtgcccctgggtgagagtgtcaccctgctgctgatccagccccagtgcgcctcagatctcgacagggtggaggtcctcgtcttccagcacgacttcctgacttggataaagaacccgcctcctcggtaggaagcatgtgggacccttgggaagtagatttgcatccaatgcacgctcacacgtcagcctgtccaggtctgtcctgtcaccacccctcccacatcctgccatcgggagcagtgtggttgcgggtatggctgttatctgtgaggttacggatactaccttcctccacggggaggcaaggacccaactggggctgtttttagggttaatatttgctgcaaggaagcatcgtgaccaaaatgcaagttggggaggaaagggtttatctggtttacacttctagatcatagcccatcactgaaggaagacaagacgggaactcaaacggcaggaacctgggagcaggagctgatgcagaggccatggaggggcgctgttcactgacttgttcctcctggcttggccagcctgctttattatagaaacaggaccaccattccagggtggaaccacccactatgggctggcccctccccccatcaatcattaattaagaaaatgccctacaggcctgcctacagcctgatcttaaaggaggcgctctcttagttgagactctagcttgtgtcaagttgacataaaactatccagcaggggtgggcactatctcattagcattttggtttttagagagcatctagagtttgtggtaattcccccacctctgcttctcaagcgcctggatgatagggttgaggtgctctgtctgtcaggctaatagccagttactcaaaagcatctttggcttttccagggaaggtgttcctcacgcagccgggctgagaacgaagggcagacagctgcccaccagccccgagctctccacagtagatgttccaggttctcctcctgtttcaggaggttttgttgtgtttgtttccaagaagttccttggggtgacaacgtcctgtgttgtgtggcccgtctgggcatatacgcaagcaaatatgtattcaccccagatagggcagggacttcggagcgaagaatcaattccctccgagtaggattcggtcattcagaacattcattgtggttacctccgtgaggcagcatgggcgaccacccgtggaagctgcatccttggagctccctgaacaacctgaggcatctctaccagtcctcctaccccatccactatttcctgtttatacaattcggggaggaacccgtggctctcccagcttccagatgtgtcttatgcaactgtttcaactcgggacacagctgcacaacactggctggctgagcagttactctcctttctcagtgggtgggagctccccagcaccctaaaggcattatctccacagggccatccgtctgaccctgccgcagctggaaattcggggatcctacaacctgcaggacctgctggctcaggccaagctgtctacccttttgggtgctgaggcaaatctgggcaagatgggtgacaccaacccccgagtgggagaggtgagtgctgccctggctgtgtctcaggggagggcgggcaggcggatggccacatggaggaagctatgcgcagggtcagcagggcaggaaggaaagagcaggcccagtaggcactgccagggctctctggctcccagtggcatgggtcacagcagtctccctcagctctcttggcatctctgttcctgcttagtccagctttgcatcctccacgaagctgaggtagattaggacggggatgcctcgatgccctgatctgcaacttctttggagagcatctctgcagaagacagagaactatgtaaaacagaggaggtgtctggatactgttgacgttgcaaaggatgctgggaaacagaggggagtcactagagaaagcatggccccgccgtgctgattagccaaatattggagagtcccctcaagatggcaggtggagacggggcacgctgaacaggggcacgacacccaggtgggagctgggtgtcggtgcgcagcccccacgtagaggacagtgtgtacacattgccttctggcaggctggacagcgtcccccctcggggcagttgagcccagtccctgtcctgtaatccctcctgaaggagagatgtctctgctttgcaggttctcaacagcatcctccttgaactccaagcaggcgaggaggagcagcccacagagtctgcccagcagcctggctcacccgaggtgctggacgtgaccctgagcagtccgttcctgttcgccatctacgagcgggactcaggtgcgctgcactttctgggcagagtggataacccccaaaatgtggtgtgatgcctcctgtgtagccatggagacaaggccagcgtcagagagctatcctgggcaaaaatcagtgccttcacccctggcttcccgtcactccttccagcaaggcagaggccgtctccttggagatggcgctaactgagaataaatgatgagcagcagcctcctggggtgtgggtttgtttggacactggggtgagagccaggagctggcactctgtataggaggactgccatcctggaaaaaaaaaatggaccaaacaactgtttgtgaaataaaaaaaaaaaaattccctttttatttgagaacacaaagtgggttttaacattaaaatgcacactgtccccttgttttgggtttgcaattagctgagtgtgagaccacgacctccgagtcccctatgccctgagttcctatccctggcaccactcattcctcacttagctgagagttcctctcagtggtaaactcagacatggtgttaccactggcagaacagacaagctagtttccctgacagactgagcaatgtgactgcagctcttgacagggaggccaacgtatgctggctgggtggtggacgagagggctgtcactaagtggcactgagagacgacctgggcggagggggcttcctttatatgtgtccaaagaccacacagggttgctactgcttctctacctgtccgcttgagattaaacctacagcttccagatctgggactgtgctgaggaaggagcgagacagggtgaggtagtggtgtgtctgtgcgtgtctgtgtgtgtacgtgtctgtgtgtacgtctctctctctctctgtgtacgtgtgtgtgtgtgtgtgtgtgtgtgtgtgtgtgtgtgtgtgtgtgtgtgtgagggtgccttcctccctgtacaaactgggaagcaggtgaacaatgtagtgtgttaaccacaggaaaagaccagagtgcctatagaatccacagggcacagacagtgggaaatcctgtattaaaacatggagttgcctctctggcctaagctggagttttgactctgcatgagcttagcctaaccttgaaggcagggctgctctgaagcttggattgagactagagggtcctgtaaggacaggtagagcctagaacatgttcctgtgaccgaggtccacccggggttgggggtggagcttaagacggcctgccactcccagtgaacacacacacatggacaccacaccacacccacatgggggtcgggggagaggctgggactggtgtacagctcactttcttagcccacgggatgctgccgtcaagtcttaagaaggttccccacatctcttcgtcccacatagaaaccctcatacatgcccagggctttgtttcttggatgactgaattccatcgggctgacaagacgagctgtcataggagtctgtgggagacagtagcaaatggacatccggccaacattagggttagtccttaccaaaggctactgtctcctacagcagccaggcttgccaagggttggttgttccccactcctcgctatacacttgagtctggcctgcaccgtcttgctaaccagcctggtgtcagtgccctggtgggacacagcacagctgagctctgtctgtgactgcgctggaagccagcacaagtcctgctgctcacagagagccgccgtcctcggcatgagcagctcgtccaccccagtgctgctggcttggcccctcagccatgggtcctcccaatggagaacagcttgaaccacaggtggtagtaaggacccgcaggcccttaggggaggtgacaacacacattgattttgaacttctgcctctacgacagaatcatttcttcccaatcagctgccatatttgcggtcactgaaagccgagtgagaaatgaatgcatgtcctttgactacataggagggagatggtgggcttcaggtccctgaatgacagactggcctcctcctcacaggcttctcatactggaaagcagtaaactgcgtggagacgggtttttagtgtgaatgttgaagctgggagaatgtgtaacttcaactttaaacctgtctgaataaaactggctctcagcggggaaaccaaggaaaggtaacaagggagctgttttggggagccagattcaagtctgagaccgttagcttttgcctgaacaacctgctctcaaggacagagacatcttagggagactggaattcaatggtttggagtcagagccaacagggtaagagaggcccactcttgacctct

SEQ ID NO: 17 - DNA, 20 bp, Artificial Sequence
  Synthetic oligonucleotide
cactgatttttgcccaggat

SEQ ID NO: 18 - DNA, 21 bp, Artificial Sequence
  Primer
agcacgacttcctgacttgga

SEQ ID NO: 19 - DNA, 21 bp, Artificial Sequence
  Primer
ttgtaggatccccgaatttcc

SEQ ID NO: 20 - DNA, 20 bp, Artificial Sequence
  Probe
aacccgcctcctcgggccat

SEQ ID NO: 21 - DNA, 21 bp, Artificial Sequence
  Primer
gctttggacgaatcttgctca

SEQ ID NO: 22 - DNA, 16 bp, Artificial Sequence
  Primer
tccccgctcctccagg

SEQ ID NO: 23 - DNA, 22 bp, Artificial Sequence
  Probe
aaaatgccctcggtccgggaaa